(19) United States
(12) Patent Application Publication
(10) Pub. No.: US 2022/0370557 A1
(43) Pub. Date: Nov. 24, 2022

(54) RECOMBINANT FASCIOLA HEPATICA FATTY ACID BINDING PROTEIN (FH15): AN ANTI-INFLAMMATORY BIOTHERAPEUTIC

(71) Applicant: UNIVERSITY OF PUERTO RICO, San Juan, PR (US)

(72) Inventors: Ana M. ESPINO, San Juan, PR (US); Jose J. ROSADO-FRANCO, San Juan, PR (US)

(21) Appl. No.: 17/842,599

(22) Filed: Jun. 16, 2022

Related U.S. Application Data
(63) Continuation-in-part of application No. 16/280,999, filed on Feb. 20, 2019.
Application No. 16/280,999 is a continuation-in-part of application No. 15/156,284, filed on May 16, 2016, now Pat. No. 10,376,561.
(60) Provisional application No. 62/632,998, filed on Feb. 20, 2018.
(60) Provisional application No. 62/161,582, filed on May 14, 2015.

(51) Int. Cl. A61K 38/17; A61P 31/04

(52) U.S. Cl. CPC A61K 38/1767; A61P 31/04

(57) ABSTRACT

The present disclosure reports that (1) recombinant Fh15 significantly prevented bacteremia, suppressed LPS levels in plasma and the production of C-reactive protein and procalcitonin, which are key signatures of inflammation and bacterial infection, respectively; (2) reduced the production of pro-inflammatory cytokines; and (3) increased innate immune cell populations in blood, which suggests a role in promoting a prolonged steady state in rhesus macaques even in the presence of inflammatory stimuli. This is the first report demonstrating that a F. hepatica-derived molecule possesses potential as anti-inflammatory drug against sepsis in an NHP-model. Prophylactic effects of rFh15 administered in a non-human preclinical primate model of sepsis support its use as a biotherapeutic.

### GOVERNMENT INTEREST

This invention was made with government support under grant numbers 1SC1AI155439-01, (ORIP-NIH) 2016-2021, P40OD12271, 5R25GM061151, G12MD007600, and R25 GM061838, awarded by The National Institutes of Health (NIH). The government has certain rights in the invention.

### CROSS REFERENCE TO RELATED APPLICATIONS

This application is a Continuation-in-Part of U.S. patent application Ser. No. 16/280,999, filed Feb. 20, 2019, which claims the benefit of priority under 35 USC § 119(e) to U.S. Provisional Patent Application No. 62/632,998, filed Feb. 20, 2018, and which is a Continuation-in-Part of U.S. patent application Ser. No. 15/156,284, filed May 16, 2016 (now U.S. Pat. No. 10,376,561); and U.S. patent application Ser. No. 15/156,284 claims the benefit of priority under 35 USC § 119(e) to U.S. Provisional Patent Application No. 62/161,582, filed May 14, 2015. The disclosures set forth in the referenced applications are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jun. 13, 2022, is named 71900-352099_SL.txt and is 5,368 bytes in size.

### BACKGROUND OF THE INVENTION

Sepsis caused by Gram-negative bacteria affects 1.7 million adults annually in the United States and is one of the most important causes of death at intensive care units. Although the effective use of antibiotics has resulted in improved prognosis of sepsis, the pathological and deadly effects have been attributed to the persistent inflammatory cascade of sepsis. There is a present need to develop anti-inflammatory agents that can suppress or neutralize the inflammatory responses and prevent the lethal consequences of sepsis.

Fatty acid binding proteins (FABPs) in platyhelminths constitute a multigenic family of cytoplasmic proteins with isoforms localized in tegument and parenchymal cells. Parasitic trematodes are unable to synthesize lipids de novo, in particular long-chain fatty acids and cholesterol. Therefore, they use carriers to uptake such lipids directly from the host and transport them to specific destinations within parasite, a process in which FABP could play an important role. Native or recombinant variants of F. hepatica FABP (Fh12 and Fh15) have shown to induce cross-protection in a variety of experimental animals including mice, rabbits, and sheep. Thus, Fh12 or Fh15 are antigens with potential for vaccine development against F. hepatica or S. mansoni infections. To induce protection against these parasites, Fh12 or Fh15 needs to be injected in animals by subcutaneous route emulsified in any type of adjuvant. In experimental conditions Fh12 and Fh15 elicit high antibody levels specifically of the sub-class IgG2a, which has been correlated with reduction of parasite burden, anti-egg fecundity and decreased liver damage. However, despite that Fh12 and Fh15 have been tested in the past as potential vaccines against Fasciola or Schistosoma spp., no studies have reported that these molecules could exhibit strong anti-inflammatory properties and serve as drugs against the sepsis caused by LPS, the potent endotoxin of Gram-negative bacteria.

### SUMMARY OF THE INVENTION

Due to their phylogenetic proximity to humans, non-human primates (NHP) are considered an adequate choice for basic and pre-clinical model of sepsis. Gram-negative bacteria are the primary causes of sepsis. During infection bacteria continuously release the potent toxin lipopolysaccharide (LPS) into the bloodstream, which triggers an uncontrolled systemic inflammatory response leading to death. Using a mouse model of septic shock, previous research has demonstrated in vitro and in vivo that Fh15, a recombinant variant of the Fasciola hepatica fatty acid binding protein, (hereinafter rFh15) acts as an antagonist of TLR4, suppressing an LPS-induced pro-inflammatory cytokine storm.

The present disclosure demonstrates that a low-dose of rFh15 is effective to suppress the cytokine storm and other inflammatory markers appearing during the early phase of sepsis induced in rhesus macaques (aka monkeys) by i.v. infusion with lethal doses of live E. coli.

rFh15 is expressed as a fusion protein with 6×His-Tag at the amino terminus in a novel bacterial expression system using Bacillus subtilis, which is a non-pathogenic gram positive bacterium that does not produce LPS.

Intravenous isotonic infusions of rFh15 administered 30 minutes prior to challenge with intravenous lethal doses of live E. coli suppressed bacternia, LPS levels in plasma, the cytokine storm and other markers of inflammation during the early phase of endotoxemia in rhesus macaques.

Among the novel results noted were: (1) rFh15 significantly prevented bacteremia, suppressed LPS levels in plasma and the production of C-reactive protein and procalcitonin; which are key signatures of inflammation and bacterial infection, respectively; (2) reduced the production of pro-inflammatory cytokines; and (3) increased innate immune cell populations in blood, which suggests a role in promoting a prolonged steady state in rhesus macaques even in the presence of inflammatory stimuli. This is the first demonstration that a F. hepatica-derived molecule is a candidate as an anti-inflammatory drug against sepsis in a non-human primate (NHP) model.

Disclosed herein is that a small molecule of 14.5 kDa (rFh15), can suppress bacteremia, in vivo in a mammal, endotoxemia and many other inflammatory markers in a rhesus macaque model. These results reinforce the notion that rFh15 constitutes an excellent candidate for drug development against sepsis.

Another aspect of the present claimed invention is that it relates a recombinant protein (rFh15) that is capable of being purified on a large scale. This is an improvement over prior art native F12 which was not a single isoform able to be purified on a large scale, but rather a protein complex of at least 8 isoforms. (Espino et al. 2001) The protocol to purify Fh12 takes a long time, produces low yield, therefore is not cost-beneficial and is unsuitable for scale up as an industrial lead. In contrast, the present invention provides rFh15 purification by recombinant methods amenable to commercial production. (Espino et al. 2001)

Further, “native” Fh12, a protein complex of at least 8 isoforms, has identical molecular mass, but different isoelectric points and immunological differences than rFh15. Other differences were observed between native (nF12) and the recombinant molecule denoted rFh15. Unpredictably, rFh15 may be one of the less immunogenic or immune protective members, or both, of the nFh12 protein complex. The relationship between effects of rFh15 and nFh12 was not predictable. For example, immunological differences were observed between native (nFh12) and the recombinant molecule denoted rFh15. In rabbits infected orally with F. hepatica, antibodies to nFh12 appeared by the second week postinfection whereas antibodies to rFh15 appeared much later, by 6 week postinfection.

These and other results suggested that rFh15 “could be one of the acidic forms of nFh12, and that it, in fact, may be one of the less immunogenic or immunoprotective members, or both, of the nFh12 protein complex.” (Espino et al. 2001)

In the background, the authors report
Several immunoprophylaxis studies have repeatedly shown that the purified native molecule (nFh12) always induces higher levels of protection against F. hepatica than does the recombinant (rFh15) one (Muro et al., 1997, Lopez Aban et al., 1999). This suggests that the 2 proteins may be slightly different.

Procedures for nFh12 purification at the time of publication of the 2001 report, did not allow the identification or the presence of the different isoforms.

In addition, because the claimed rFh15 can be purified on a large scale, in a different process than Fh12, that process has unpredictable effects compared to crude Fh12. As an example, the suppression of stimulation of various TLRs in response to various whole bacterial extracts was not impaired by anti-F12 antibodies, which would have been predicted.

Fh15 expression has been further optimized by using the pT7M vector, producing rFh15 as a fused protein with His-Tag in a Bacillus subtilis expression system. The recombinant variant offers ease of production and purification advantages over the native FABP, being more suitable and convenient for scale-up production.

The present technology demonstrates prophylactic effects of Fh15 in a superior pre-clinical model, namely a non-human primate sepsis model.

Because E. coli is the first choice of a host when a protein has to be expressed in vitro, a recombinant FABP termed rFh15 expressed in E. coli was initially used to determine whether Fh15 is able to mimic the anti-inflammatory properties showed by Fh12.

This Fh15 recombinant version of FABP constituted an excellent alternative in contrast to the purification of the native molecule, and it exerted a broader suppressive effect on the activation of various TLRs.

Results obtained demonstrated that Fh15 displayed a similar capacity than that of Fh12 to suppress the expression of IL-1β and TNFα induced by LPS in murine macrophages and THP1 Blue CD14 and also was able to therapeutically prevent the cytokine storm in mice exposed to lethal doses of LPS.

Fh15 also suppressed the TLR4 stimulation as well as various other TLRs induced by whole attenuated bacteria strains, specifically Klepsiella pneumoniae and Enterococcus faecalis. Importantly, the Fh15 effect was not impaired by a thermal denaturing process or blocked by the presence of anti-Fh12 antibodies.

A single intraperitoneal injection of 50 ug Fh15 (without any type of adjuvant) applied to BALB/c mice 1 h after animals were exposed to a lethal dose of LPS (7 mg/kg body weigh), was enough to suppress the cytokine/chemokine storm and prevent the septic shock in the animals. Results confirmed that Fh15 also targets the CD14 co-receptor, which impede the LPS-TLR4 binding and this stops the TLR4-signaling cascade at the beginning of the LPS-stimuli. A novel finding was that Fh15 also prevents the migration of large macrophages from the peritoneal cavity of animals exposed to LPS, thus, prolonging the steady state in the peritoneum and the excessive and decontrolled inflammation caused by LPS. Moreover, Fh15 also down regulates the expression of CD38, a receptor closely related to the classical activation of macrophages induced by LPS.

These initial results in mice were strengthened the results using B. subtilis as the host for purified rFh15, and rhesus macaques as the subject mammal.

The rFh15 construct used in the experiments on mice in an E. coli system is different from the construct used to produce the rFh15 for the experiments on monkeys in a B. subtilis system. Constructs using the B. subtilis as host are less likely than those using E. coli to have LPS contamination, which may be preferred to compositions of matter including rFh15.

### BRIEF DESCRIPTION OF THE DRAWINGS

The patent or application file contains at least one drawing executed in color. Copies of this patent or patent application publication with color drawings will be provided by the Office upon request and payment of the necessary fee.

Further features and advantages of the invention will become apparent from the following detailed description taken in conjunction with the accompanying figures showing illustrative embodiments of the invention, in which:

FIGS. 1A-1D. Levels of antigenemia in plasma samples from rhesus macaques treated with rFh15. A homemade sandwich ELISA was optimized to detect circulating rFh15 in plasma samples; The assay uses a rabbit anti-Fh15 IgG antibody as capturing antibody and a rabbit anti-Fh15-IgG conjugate with HRP as detecting antibody; Plasma samples were collected from rhesus macaques at different time points: 0 (baseline), 30 min, 2 h, 4 h, 6 h and 8 h during the experiment; (1A) The group termed Fh15 included monkeys (n=3) that only received the i.v. infusion with 15 mg Fh15; (1B) The group termed Fh15-E. coli included animals (n=3) that received the i.v. infusion containing 12 mg Fh15 followed by the E. coli infection (live E. coli 10¹⁰ CFU/kg body wt.); (1C) The group termed Fh15-Fh15-E. coli included animals from the Fh15 group that 3 months later received a second i.v. infusion with 12 mg Fh15 followed by the E. coli infection; (1D) is a composite of A-C showing antigenemia.

FIGS. 2A-1 to 2D-4. rFh15 decreases bacteremia, endotoxemia, C-reactive protein and Procalcitonin in rhesus macaques during an acute lethal sepsis. Rhesus macaques were allotted into three experimental groups (n=3) each. (1) The group termed E. coli is the control group of monkeys that only received a lethal i.v. infusion with live E. coli 10¹⁰ CFU/kg of body wt. (2). The group termed Fh15-E. coli included monkeys (n=3) that received the i.v. infusion containing 12 mg Fh15 followed by the E. coli infection. (3) The group termed Fh15-Fh15-E. coli included animals from a group that had received a primary i.v. infusion with 12 mg Fh15; (4) Group is a composite, three months later these monkeys received a second 12 mg Fh15 treatment followed by the E. coli infection. The graphs represents the levels of (2A-1 to 2A-4) viable bacteria for each single monkey of each experimental group as well as also the comparison among the significant reductions in bacteremia showed by rFh15-E. coli and rFh15-rFh15-E. coli compared to the E. coli control group (***p=0.0001). (2B-1 to 2B-4) levels of plasma endotoxin (LPS) (***p<0.0001), (2C-1 to 2C-4) C-reactive protein (***p=0.004), and (2D-1 to 2D-4) procalcitonin (****p<0.0001). Statistical significance between control group and experimental groups was determining by ANOVA or Student t-test using GraphPad Prism-8. For all tests, a p value <0.05 was considered significant.

FIGS. 3A-3F. Fh15 tends to decrease the production of pro-inflammatory cytokines and chemokines in rhesus macaques undergoing E. coli infection. Plasma samples were collected from rhesus macaques at different time points: 0 (baseline), 30 min, 2 h, 4 h, 6 h and 8 h. E. coli: included animals that only received the i.v. infusion with live E. coli (10¹⁰ CFU/kg body wt.), Fh15-E. coli: included animals that received an i.v. infusion containing 12 mg Fh15 followed by the E. coli infection and Fh15-Fh15-E. coli: included animals from the Fh15 group that 3 months later received a second i.v. infusion with 12 mg Fh15 followed by the E. coli infection. Levels of (3A) IL-6, (3B) TNFα, (3C) IL-12, (3D) IFN-γ, (3E) IP10 and (3F) MCP-1 were measured using Luminex technology. Animals from Fh15 group do not elicit pro-inflammatory cytokines.

FIGS. 4A-4H. Effect of live E. coli compared to rFh15 treatment on the number of innate immune cells in the bloodstream. Graphs represent the number of (4A) peripheral blood monocytes, (4B) classical monocytes, (4C) non-classical monocytes, (4D) intermediate monocytes, (4E) dendritic cells, (4F) plasmacytoid DCs, (4G) natural killer cells and (4H) neutrophils, over time, in four experimental groups: E. coli: rhesus macaques (n=3) that received an i.v. isotonic infusion with live E. coli (10¹⁰ CFU/kg body wt.); Fh15: rhesus macaques (n=3) that only received i.v. infusion with 12 mg Fh15; Fh15-E. coli: rhesus macaques (n=3) that received the Fh15 infusion followed by the E. coli infection; Fh15-Fh15-E. coli: rhesus macaques (n=3) from the Fh15 that three months later received a second infusion with 12 mg Fh15 followed by the E. coli-infusion; Whole blood samples were stained with a cocktail of antibodies for labeling surface markers specific for peripheral monocytes (CD14⁺, CD16⁺), myeloid and plasmacytoid dendritic cells (CD11c⁺, CD123⁺), natural killer cells (HLA-DR⁺, CD3⁻, CD69⁺) and neutrophils (CD66a/c/e⁺); Cells were analyzed by gating using a MacQuantlO. Data was analyzed using FlowJo. Data represent average number of cells±SD of three independent biological samples, each in duplicate at every time point studied.

FIGS. 5A-5B. Purity of Fh15. Recombinant Fh15 was successfully expressed within Bacillus subtillis as fusion protein with 6His-Tag at the amino terminal. (5A) SDS-PAGE and (5B) Western Blot were used to assess the purity of the protein; M1 represents a BSA (2 μg) as protein control, lane 1 represents Fh15 (2 μg) under reducing conditions and lane-2 represents Fh15 (2 μg) under reduced conditions incubated with a mouse antibody against the histidine tag.

FIGS. 6A-6D. Schematic representation of the experimental design disclosed herein. Diagrams represent the experimental design performed with rhesus macaques; Experimental groups (n=3) included naïve male rhesus average weighting ˜8.46 kg; Animals were allotted into four groups; (6A) The control group termed E. coli, only received an i.v. isotonic infusion (50 -ml) containing a lethal dose of live E. coli (10¹⁰ CFU/kg body wt.); This infusion was administered by 2 h at a flow rate ˜0.416 ml/min; (6B) Another group termed Fh15 only received the isotonic infusion (5-ml) containing 12 mg Fh15. This infusion was administered during 20 min at a flow rate ˜0.25 ml/min; (6C) The group termed Fh15-E. coli received first the Fh15-infusion followed by the E. coli-infusion; (6D) The group termed Fh15-Fh15-E. coli included the same animals from Fh15 group, which 3 months after receiving the first Fh15 infusion were returned to the experiment to receive a second Fh15-infusion+E. coli infusion. Blood samples were collected at baseline (0 min), 30 min, 2 h, 4 h, 6 h and 8 h. All animals that received the E. coli infusion were euthanized at 8 h; Blood samples collected were used for determining levels of bacteremia and then staining with two antibody cocktails specific for cell markers of innate immune cell populations; These samples were analyzed by flow cytometry; Plasma recovered from the blood samples was used for measuring levels of LPS, antigenemia, CRP, PCT and cytokines/chemokines. Colors are used to match results of diagnostic tests with monkey body parts: brown=viable bacteria content; red=plasma levels of antigenemia, levels of LPS, cytokine/chemokine panels, CRP, PCT. Flow cytometry was used.

FIGS. 7A-7B. Diagram of the recombinant fusion protein: (7A) amino acid sequence (SEQ ID NO: 13) and (7B) nucleic acid sequence (SEQ ID NO: 14). The yellow color indicates an Ndel, red shows the stop codon, and gray the Hind III. The plasmid is vector pT7M.

### DETAILED DESCRIPTION OF THE INVENTION

As part of their immunomodulatory mechanisms, helminths establish a regulatory anti-inflammatory immune response in their mammalian host with a prominent T helper-2/T regulatory (Th2/Treg) immune profile, which is thought to be mutually beneficial for host and parasite, because it protects the host from severe consequences of inflammatory responses while preventing the elimination of worms. Thus, human and animal studies have demonstrated that helminth infections could be used to ameliorate or prevent inflammatory diseases. In fact, severe sepsis is significantly less frequent in persons carrying chronic helminth infections than it is in non-parasitized persons.

These studies have helped incorporate helminth infections into the expanded ‘Hygiene hypothesis’. Fasciola hepatica, one of the most prevalent parasitic Platyhelminths, is not an exception. However, because of the pathogenicity and negative impact that F. hepatica exerts on the health of animals and humans, the infection with this parasite cannot be used to treat inflammatory diseases in humans. Additionally, the immune regulation associated with F. hepatica infection lacks specificity and results in a compromised immune system unable to respond effectively to bystander infections. It more judicious to identify and purify defined immune-modulatory molecules produced by the parasite, which have the potential for drug development and to characterize their precise mechanism of action. Owing to its extraordinary capabilities for the host's immune-modulation, F. hepatica constitutes an enormous ‘pharmacopeia’. As soon as the parasite invades the gut wall, it initiates a complex interaction with various host immune cells (e.g. macrophages or dendritic cells). The parasite secretes a myriad of immunomodulatory molecules termed excretory-secretory products (ESPs) that direct the host's immune response toward a non-protective Th2/Treg environment with suppressed Th1 immunity, which allows the parasite to persist in the host for a long period of time. Some of these molecules belong to the fatty acid binding protein family (FABP).

F. hepatica FABPs are antioxidant molecules that have been extensively used as vaccine candidates against fascioliasis or schistosomiasis. In previous work, a single therapeutic dose of recombinant F. hepatica FABP (Fh15; 50 μg) given to mice 1 h after exposure to lethal LPS challenge significantly suppressed pathological sequelae by concurrently modulating the dynamic of macrophages in the peritoneal cavity and the activation status of spleen macrophages in a mouse model of septic shock.

Because non-human primates (NHP) share physiological and anatomical features similar to humans and have shown to respond similar to humans when exposed to live Gram-negative bacteria, they represent a more relevant pre-clinical models than rodents-models to study the inflammatory responses during the acute phase of sepsis. With these advantages in mind, a previous study reported a rhesus macaque model of septic shock induced by intravenous (i.v.) administration of live Escherichia coli to identify inflammation-associated markers during the early phase of sepsis in rhesus macaques. Bacteremia was present in all animals from 30 minutes to 3 hours following E. coli infusion, whereas endotoxin, C-reactive protein (CRP) and Procalcitonin (PCT) were detected during the full-time course suggesting an ongoing inflammatory process caused by an active bacterial infection. Similarly, TNF-α was detected at 2 h whereas IL-6, IL-12, and IFN-γ were detected after 4 h of E. coli infusion.

The present disclosure addresses whether these inflammatory markers can be suppressed when a single low dose of Fh15 is administered to a NHP i.v. as isotonic infusion 30 min before a challenge with live E. coli-infusion. Knowing that Fh15 is a protein molecule, and its development as drug could be hampered by problems owing to limitations such as half-life and immunogenicity (capacity to induce the formation of anti-Fh15 antibodies) the present disclosure also shows the duration of Fh15 in circulation as well as determining whether its immunogenicity could hamper the anti-inflammatory effect.

Some advantages of rFh15 over existing technologies e.g. nFh12 treatment:
Fh15 expression optimization results in higher yields and easier purification than native Fh12, which is a mixture of isoforms.
Fh15 anti-inflammatory capacity has been demonstrated in multiple models in vitro and in vivo.
Fh15 dramatically suppresses the TLR-stimulation induced by Gram-positive or Gram-negative bacteria extracts in THP1 Blue CD14 cells.
The present prophylactic studies in non-human primates (NHP) provide a strong preclinical relevance due to their higher anatomical and physiological similarities with humans.
Similar to the natural form, Fh15 alone does not induce the host's inflammatory responses. It does not induce IL-12, IL-6, IFNγ, TNFα, IP-10 or MCP-1 in non-human primates.
The presence of anti-Fh12 antibodies does not impair the capacity of Fh15 to suppress the cytokine storm in the rhesus macaques.

Some disadvantages of rFh15 over existing technologies.
Fh15 produced in Bacillus subtilis, another prokaryotic expression system, other than E. coli is unable to undergo post-translational modifications due to the lack of organelles required for such modifications in higher eucaryotic systems. In vitro studies have demonstrated that a two-fold increase in concentration of the recombinant form Fh15 (expressed in E. coli) is needed to produce an effect comparable to that of the natural form Fh12 on TLR-stimulation and the expression of inflammatory cytokines.
Gram-positive bacteria such as Staphylococcus aureus and Streptococcus pyogenes are among the most common causes of sepsis.

### Physiological Parameters Before and After Sepsis Induction

At baseline, BT, HR, and RR of all rhesus macaques involved were at normal values ranging between 33.4-37.2° C. (median 35.9° C.), 85-160 beats/min (median 114 beats/min) and 13-25 breaths/min (median 21 breaths/min), respectively. The MAP ranged between 33-78 mmHg (median 51 mmHg). These MAP measurements were lower than expected. According to recent literature oscillometric monitoring of BP are often underestimated. None of the monkeys in the experiment died as a result of the experimental treatment. Monkeys that received the E. coli infection were euthanized at 8 h of experimentation. At the conclusion of the experiment the physiological parameters were similar or slightly higher than the baseline for most of animals (see Table 3).

For experimental reasons explained in the Material and Methods section, monkeys in the Fh15 group were allowed to recover after 8 h. Therefore, it was not possible to determine by postmortem examination whether the Fh15 dose administered induced any gross type of toxicity. However, the physiological parameters of these animals remained at normal values during the entire time course of the experiments and after recovering and returning to their respective cages, they remained healthy for 3 months (Table 3). One of these monkeys (MA035) when it received the second Fh15 treatment followed by the E. coli infection (group Fh15-Fh15-E. coli) experienced a MAP dropped from 41 to 21 mmHg and the RR dropped from 20 to 5 breaths per minute (bpm). Despite this abrupt drop of RR, the monkey did not die prematurely. Postmortem examination of monkeys revealed no gross abnormalities with exception of splenomegaly, which was noticed in MA035, and lymphadenopathy that was observed in all monkeys from the E. coli group.

### Developing of Antibodies to Fh15 and Dynamic of Fh15-Antigenemia

A double antibody sandwich ELISA optimized as described in the Materials and Methods was used to measure levels of circulating Fh15 in plasma of Fh15-infused rhesus macaques. Antibodies against Fh15 were measured by a previously optimized indirect ELISA (27). As expected, none of rhesus macaques in the Fh15 or Fh15-E. coli groups had antibodies or circulating Fh15 in the plasma samples collected at baseline, which confirmed that none of animals had been previously exposed to this antigen or were infected with parasites that potentially could induce cross-reactive antibodies to Fh15.

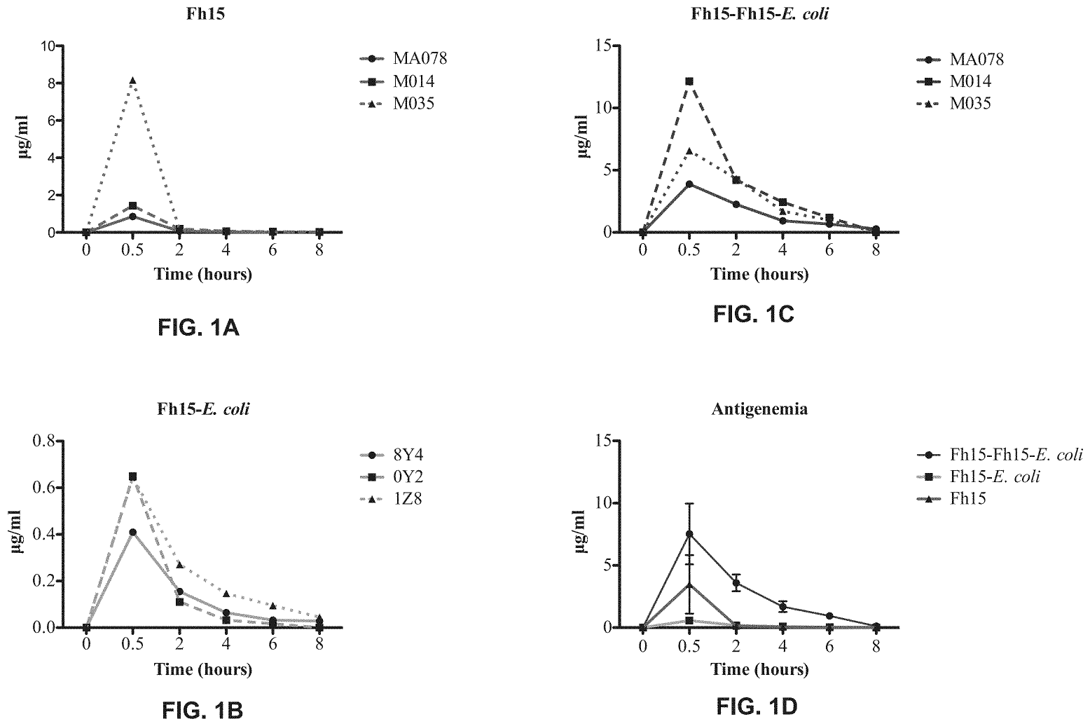

When the plasma samples collected from animals in the Fh15 group (MA014, MA035 and MA078) were tested for antigenemia, maximal Fh15 concentrations (3.48±3.31 μg/ml) at 30 min following the infusion, which at 2 h declined to an average of 0.135±0.048 μg/ml were undetectable at subsequent time points. Monkey M035 was the main contributor to this antigenemia with an average concentration of 8.16±0.3 ug/ml at 30 min. In the monkeys from Fh15-E. coli group (8Y4, 0Y2, 1Z8) the average Fh15 concentration at 30 min was 6.12-fold lower than in the Fh15 group (mean 0.568±0.11 μg/ml). At 2 h the antigenemia declined in this group to an average of 0.179±0.067 μg/ml and was undetectable thereafter. The plasma samples collected prior to the second Fh15 infusion administered to animals from Fh15-Fh15-E. coli group (rhesus macaques MA014, MA035, and MA078) tested positive for antibodies with titers against Fh15 among -1:200 to 1:400. The average plasma Fh15 concentration in these animals at 30 min was 7.53±3.44 μg/ml, which is 2.16-fold higher than those observed in the Fh15 group and 13.25-fold higher than the observed in the Fh15-E. coli group. At 2 h the antigenemia dropped to an average of 3.599±0.95 μg/ml, at 4 h to 1.685±0.612 μg/ml and at 6 h still was detectable with concentrations of 0.94±0.21 μg/ml. After 8 h, circulating Fh15 was no longer detected (FIGS. 1A-1D).

### Fh15 Suppress the Bacteremia, Endotoxemia and the Production of Acute-Phase Proteins

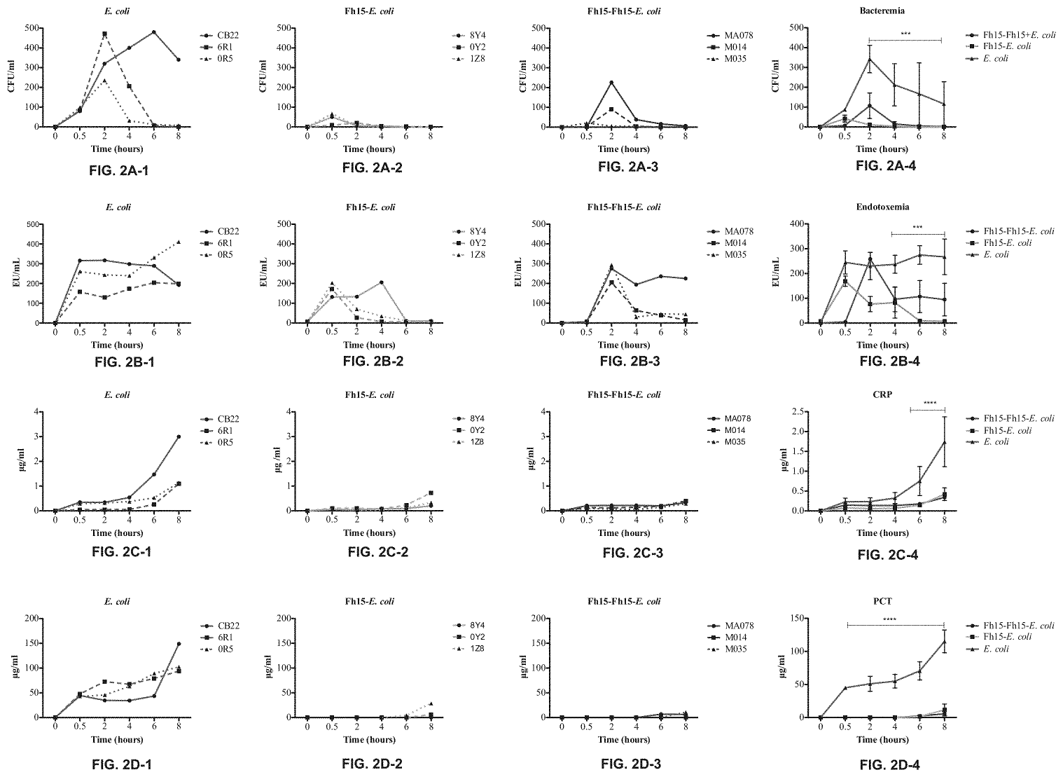

Monkeys that only received the bacterial infusion (CB22, 6R1, 0R5) had highest levels of bacteremia, which were high from 30 min following the bacterial infusion administration (87.33±7.71 CFU/ml). Overall, the bacteremia in this group increased progressively until reaching a peak at 2 h (342.66±97.67 CFU/ml). At 4 h, the average bacteremia in this group was 212.66±150.30 CFU/ml. One monkey of this group (CB22) had high and persistent levels of bacteremia at every time point (average 385±62.24 CFU/ml) whereas in the other two animals (6R1 and 0R5) the average number of viable bacteria declined dramatically to 10±2 CFU/ml and 4 CFU/ml at 6 h and 8 h, respectively (FIGS. 2A-1 to 2A-4).

The levels of plasma LPS in all animals of the E. coli group increased abruptly at 30 min (244.81±65.76 EU/ml) and remained at very high levels throughout the experiment (252.3±76.8 EU/ml) (FIGS. 2B-1 to 2B-4). The increase of plasma endotoxin is a consequence of the bacteria disruption and consequent releasing of LPS to the bloodstream. The C-reactive protein (CRP) (FIGS. 2C-1 to 2C-4) an acute phase protein that typically increases in plasma during inflammatory process was detectable at 6 h and reached maximal values at 8 h following the E. coli-infusion (1.976±1.218 μg/ml). Procalcitonin (PCT) (FIGS. 2D-1 to 2D-4) a marker of bacterial infection and a predictor of sepsis in humans was detected from 30 min and increased sequentially until reach maximal levels at 8 h following E. coli infusion (115.21±24.23 pg/ml).

The animals that only received the Fh15 infusion did not develop bacteremia, endotoxemia, CRP or PCT at any time throughout the study. This is clear evidence that the Fh15-infusion was prepared pyrogenic-free.

An important finding is that the number of blood viable bacteria in the Fh15-E. coli group was drastically lowered at every time point compared to the E. coli group (FIGS. 2A-1 to 2A-4). These monkeys had 2.079-fold lower viable bacteria (average 42±24.3 CFU/ml), 31.15-fold lower (average 11±6.37 CFU/ml) and 70.88-fold lower (average 3±2.1 CFU/ml) than in the E. coli control at 30 min, 2 h and 4 h, respectively, with no viable bacteria detected in the subsequent time points. All these reductions in bacteremia were found significant (p=0.0001).

When the bacteremia were analyzed in rhesus from the Fh15-Fh15-E. coli group, a similar pattern was observed although with some differences. In two animals (M014 and M035) the bacteremia reached maximal values at 2 h (47±35 CFU/ml) declining quickly at 4 h (4 CFU/ml) and making undetectable thereafter. In the rhesus MA078 the maximal number of viable bacteria was also detected at 2 h (226 CFU/ml), declined to 38 CFU/ml by 4 h and to 16 CFU/ml and 6 CFU/ml at 6 h and 8 h, respectively.

Statistical differences were found between the bacteremia in the Fh15-Fh15-E. coli group compared to the E. coli-group at 30 min (p=0.0488) and 4 h (p=0.0055). However, no statistical differences were found between the levels of bacteremia in the Fh15-E. coli and Fh15-Fh15-E. coli groups at any of time points studied. These results demonstrate that the treatment with Fh15 applied 30 min prior to exposure to a lethal live E. coli infusion can efficiently prevent the bacterial replication in blood.

In agreement with the decreasing of bacteremia, the LPS levels in plasma were also notably lower in both experimental groups compared to the E. coli group. In the group Fh15-E. coli group average concentration of LPS at 30 min was 169.16±29.27 EU/ml, which represents a significant lowering of 1.44-fold less LPS than in the E. coli group (p=0.0067). The concentrations of LPS in this experimental group lowered subsequently reaching the lowest levels at 8 h (7.3±2.5 EU/ml), which represented a significant diminution (p=0.0042) of 36.60-fold compared to the E. coli group (FIGS. 2B-1 to 2B-4). In the group Fh15-Fh15-E. coli the levels of LPS at 30 min were very low but at 2 h had reached a peak (average 258.33±38.11 EU/ml). In two animals (M014 and M035) the LPS concentrations reduced sequentially until reach lowest levels of 29.75±14.65 EU/ml at 8 h, which represented a significant lowering of 8.98-fold lower concentration than in the E. coli group (p=0.0224). However, in the rhesus MA078 the LPS concentrations remained consistently high throughout the experiment with an average concentration of 218.6±17.63 EU/ml.

Statistical differences between both experimental groups and the E. coli group were found at 4 h, 6 h and 8 h (p<0.0001). The Fh15-E. coli and Fh15-Fh15-E. coli groups had significantly lower levels of CRP and PCT than the E. coli group (p=0.0001) (FIGS. 2C-1 to 2C-4 and 2D-1 to 2D-4).

### Fh15 Suppress the Production of Pro-Inflammatory Cytokines in Plasma Of Septic Rhesus Macaques

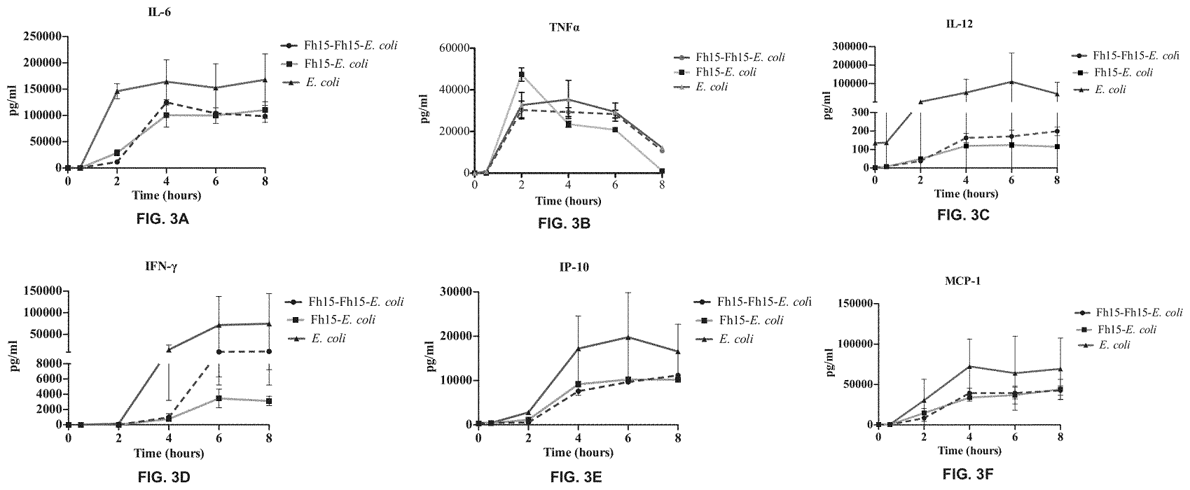

Having demonstrated that the administration of Fh15 prior to a live bacterial infusion is able to suppress bacteremia, endotoxemia as well as levels of CRP and PCT induced by E. coli, a question investigated was whether Fh15 could also suppress several pro-inflammatory cytokine/chemokines that are signatures of inflammation during sepsis. As expected, all animals from the E. coli control group developed a strong pro-inflammatory cytokine storm evidenced by the high levels of IFNγ, IL-6, TNFα, IL-12, IP-10, and MCP-1 throughout the entire time-course. This is consistent with the high levels of LPS, CRP and PCT detected in plasma of these animals, which is indicative of an ongoing bacterial sepsis. However, all animals from groups Fh15-E. coli and Fh15-Fh15-E. coli had lower concentrations of IL-6, TNFα, IL12, IFNγ cytokines and lower IP10 and MCP-1 chemokines than monkeys from the E. coli control group at every time point studied (FIGS. 3A-3F).

Unfortunately, due to the large variability found in levels of cytokines/chemokines among monkeys from a same group, no statistical differences were found. To better appreciate the reduction that produced Fh15 in the levels of these pro-inflammatory cytokines/chemokines the fold changes in cytokine/chemokine levels were analyzed as treatment-related decreased from untreated controls. For this analysis the mean concentration of each cytokine/chemokine induced by E. coli at every time point was divided to the mean concentration of cytokine/chemokine induced by the Fh15 treatment and the result was expressed as a negative value (Table 4).

This analysis revealed that animals from the Fh15-E. coli group had between 11.28 to 23.97 fold less IFNγ than the E. coli group at 2 h to 8 h, respectively. The reduction in IFNγ levels in the Fh15-Fh15-E. coli ranged among 14.95 to 7.03 at 2 h and 8 h, respectively. IFNγ is a cytokine classically produced by NK-cells and T lymphocytes, which facilitates systemic inflammation during endotoxin-induced shock. IL-12, a cytokine naturally produced by DCs, macrophages and neutrophils cells in response to antigenic stimulation was found among 20.66 to 386.18-fold reduced in the Fh15-E. coli group and 18.36 to 223.44-fold reduced in the Fh15-Fh15-E. coli group. TNFα an inflammatory cytokine produced by macrophages/monocytes during acute inflammation, which normally has a peak of secretion about 2 h following the E. coli insult with a further decline later was also found reduced among 23.34-fold to 11.42-fold in the Fh15-E. coli group and 6.57 to 1.03-fold reduced in the Fh15-Fh15-E. coli group, during the entire time course. IL-6, a cytokine produced by a variety of cells including macrophages and monocytes during inflammatory process was also found 2.33-fold and 1.52-fold reduced at 2 h and 8 h, respectively in the Fh15-E. coli as well as also there were a 2.31-fold and 1.7-fold reduced in the Fh15-Fh15-E. coli group, respectively. Similarly, the chemokines IP-10 and MCP-1, were found 5.41 to 1.48-fold or 1.76 to 1.63-fold reduced in the Fh15-Fh15-E. coli group when compared to the E. coli group (Table 4).

### Effect of Bacterial Infusion on the Innate Immune Cells Population and the Counter Effect Caused by Fh15

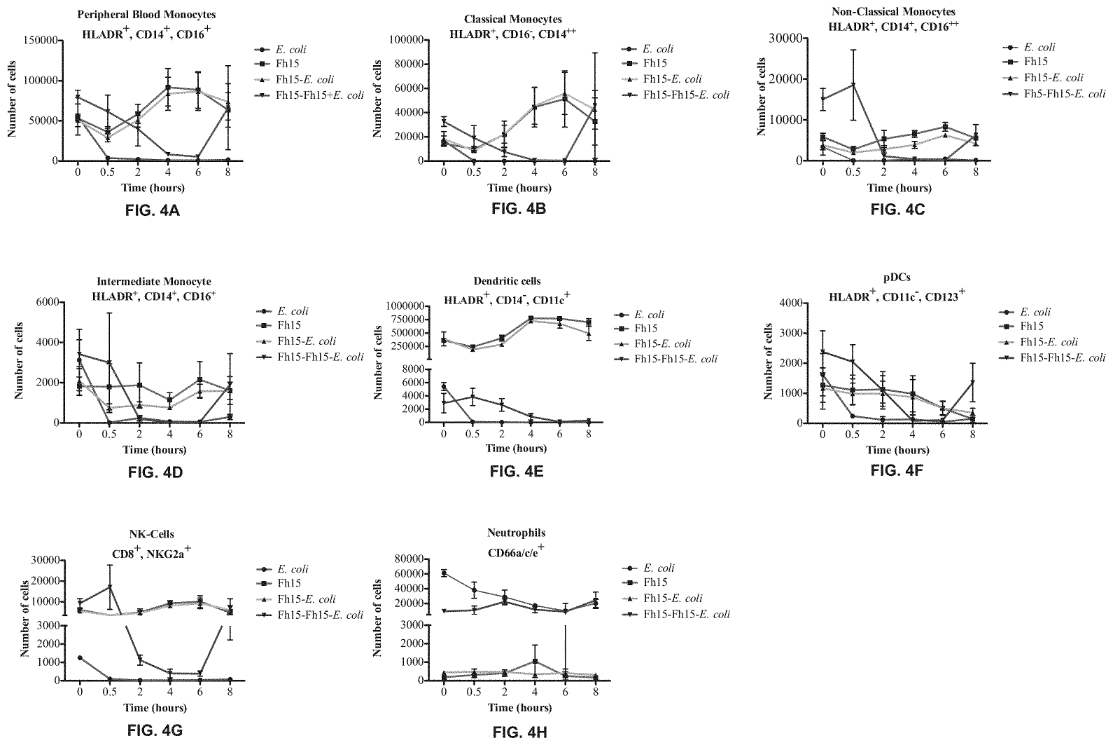

When the blood innate immune cell populations in the group that only received the E. coli infusion were examined, it was noticed that most of the cell populations had significantly decreased by 30 min following the infusion (p<0.0001) and remained at very low levels throughout the entire full-time course of the experiment (FIGS. 4A-4H). Specifically, peripheral blood monocytes dropped >14.8-fold compared to baseline. Classical monocytes, which comprise about 80-95% of circulating monocytes and are highly phagocytic had dropped >2,000-fold. Non-classical monocytes that comprise about the 2-11% of circulating monocytes (30) and have pro-inflammatory behavior had dropped >100-fold. Intermediate monocytes, which comprise about 2-8% of circulating monocytes and have among their functions the production of reactive oxygen species (ROS), antigen presentation, stimulation and proliferation of T-cells and angiogenesis (30) had dropped >130-fold. Dendritic cells (DCs) and plasmacytoid DCs (pDCs), which play pivotal roles in the initiation of innate and adaptive immune response to pathogens dropped >50-fold and >6-fold, respectively. Natural killer (NK)-cells, which make up 5-15% of human peripheral blood and play protective roles against both infectious pathogens and cancer dropped >12-fold.

Interestingly, in the group that only received the Fh15 infusion, most of these cell populations increased progressively until reaching maximal levels between 4 to 8 hours. At peak the amount of classical monocytes population was 3.5-fold higher than baseline, there was 1.42-fold more non-classical monocytes, and 1.18-fold more intermediate monocytes than in the baseline. The DCs and NK-cells populations also were found 2.13-fold and 1.64-fold more abundant than baseline, respectively. Although the number of cells decreased slightly after peak all cell populations remained at high levels until the end of the study. The pDCs and neutrophils remained at levels relatively like baseline although the trend of pDCs was to decline sequentially. Importantly, a dynamic almost identical to those described for Fh15 was observed within animals from the Fh15-E. coli group. The fh15-Fh15-E. coli group had a different dynamic of innate immune cells in which the amount of all monocyte populations, DCs. and NK-cells declined sequentially until reaching the lowest levels amount between 4 to 6 hours and then increased notably at the end of the procedure (FIGS. 4A-4H, Table 5).

Previous in vitro studies using THP1-Blue CD14 cells and mouse bone marrow-derived macrophages demonstrated that Fh15 can suppress the NF-κB activation induced by different Gram-negative and Gram-positive bacteria extracts and suppress a number of the LPS-induced pro-inflammatory cytokines, respectively. Moreover, in vivo studies using a mouse model of sepsis demonstrated that Fh15 could be useful as prophylactic and therapeutic drug to prevent the dissemination of the cytokine storm in the animals that received Fh15. The present disclosure shows Fh15 is useful limiting the lethal inflammatory responses and bacterial dissemination caused by an i.v. infusion with live E. coli in rhesus macaques.

Fh15 is an immunogenic molecule capable of inducing strong antibody response when injected subcutaneously in rabbits or mice emulsified in adjuvant. Hence, rabbits and mice infected with F. hepatica also develop antibodies against Fh15. All rhesus macaques that received the Fh15 infusion in the present study haD detectable antibody against Fh15 three months later. However, what was surprising was the observation that these same animals not only had antibodies, but also higher levels of antigenemia. In the Fh15 group the main contributor to the antigenemia was MA035, but in the Fh15-Fh15-E. coli group the three monkeys (MA078, M014 and M035) contributed similarly to the antigenemia. To rule out any technical mistake, these determinations were repeated three times in different days with similar results.

The detection of antibodies against Fh15, concurrently with circulating Fh15, suggests that the antibodies elicited against Fh15 had low affinity and did not attach strongly enough to the antigen; otherwise, the sandwich ELISA would not have detected circulating Fh15. The production of antibodies of low titer and low affinity often occurs when the exposure to the antigen has been discontinued and fail the selection of clones that produce antibody of high affinity.

The rationale to include in the present disclosure an experimental group that has antibodies specific to Fh15 was precisely to determine whether these anti-Fh15 antibodies may block the ability of Fh15 to exert its anti-inflammatory function. In that regard, it was interesting to observe that animals with similar levels of anti-Fh15 antibodies (Fh15-Fh15-E. coli group) had similar ability to those that were naïve to Fh15 (Fh15-E. coli), to prevent bacterial replication, reduce the concentration of plasma LPS and suppress the production of CRP and PCT (FIGS. 1A-1D), suggesting that at least these functions were not impaired by the presence of antibodies.

In humans undergoing sepsis or septic shock, CRP is known to activate the complement system and increase antigen presentation. Also, it has been correlated with an increased organ dysfunction, longer hospital Intensive Care Unit (ICU) stay and mortality. PCT in the ICU is used as a biomarker for survival, with lower levels resulting in lower sequential organ failure assessment (SOFA) scores and better prognosis in septic patients.

The observation that two monkeys (6R1 and 0R5) that only received the E. coli infusion had bacteremia by only 4 hours is expected. The rapid decline of bacteremia in these monkeys indicates that bacteria were unable to colonize and replicate inside these animals and were lysed by the complement system as has been reported in experimental conditions where high bacteria inoculum are used to induce sepsis. Because all animals received the same bacterial dose adjusted to the body weight, the detection of bacteremia in one animal (CB22) at every time point suggests failure in the efficiency of the complement system to lyse bacteria.

An important observation was the inherent antibacterial activity showed by Fh15. Due to the bacteremia suppression that occurred concurrently by the time in which the levels of Fh15 in plasma are maximal, it is postulated that Fh15 has inherent antibacterial activity capable of inducing immune cells to release extracellular DNA traps (ETs) to trap and kill bacteria. This process termed Etosis is a distinct process of cell death and has been reported occur in several immune cells including neutrophils, eosinophils, mast cells and monocytes/macrophages from humans or mice. Etosis has been also reported in humans and mouse immune cells either in vivo or in vitro in response to protozoa and helminth parasites infections including Toxoplasma gondii, Leishmania amazonensis and Strongyloides stercoralis.

Another interesting observation was the abrupt and sustained decrease in the blood innate immune cells during the 8 hours following to the E. coli infusion (FIGS. 3A-3F). Blood monocytes (including classical, non-classical and intermediate monocytes), NK cells and DCs are key cells during early phase of endotoxemia because they are not only responsible for maintaining vascular homeostasis, but also are highly responsible for patrolling the bloodstream to recognize and phagocyte invading pathogens, resulting in the secretion of pro-inflammatory cytokines, such as IL-1β, TNF-α, IL-6 and IL-8.

The ‘disappearance’ of immune cells from the blood stream in rhesus macaques from the E. coli-group could be a consequence of an inflammatory insult because immune cells migrate to secondary lymphoid organs to maturate, differentiate, and present the antigen to the naïve T-cells. This observation is consistent with a similar phenomenon fully documented that occurs in mice exposed to lethal intraperitoneal doses of LPS in which large peritoneal macrophages (LPMs), the macrophage population more abundant at steady state, “disappear” from the peritoneal cavity at the beginning of the LPS-insult. In that regard, that Fh15 shifts the LPMs dynamic by not only preventing the disappearance of LPMs but also augmenting this cell population in the peritoneal cavity. The observation that Fh15 alone or in the presence of E. coli can promote the persistence of innate immune cells in the bloodstream during the early phase of the endotoxemia suggests that a primary modulatory mechanism of Fh15 would be based on generating an immunological environment compatible with the homeostasis or a prolonged steady state even in the presence of inflammatory stimuli.

However, based on the results showed in (FIGS. 3A-3F), the ability for Fh15 to increase the innate immune cell population seems to be partially abrogated in the rhesus macaques exposed to Fh15 three months before and that had antibodies against Fh15 (Fh15-Fh15-E. coli). However, it was noticed that the reduction of cell populations in this experimental group was sequential and not abrupt as occurred in animals that only received E. coli, and at the end of the experimental window all cell populations had again increased. Therefore, that in these same animals the presence of antibodies does not seem to affect the decrease of bacteremia, endotoxemia, CRP or PCT or the ability for Fh15 to reduce the pro-inflammatory cytokine/chemokines (FIGS. 2A-1 to 2D-4).

The observation that Fh15 alone does not induce IL-12, IL-6, IFNγ, TNFα, IP-10 or MCP-1 is consistent with previous studies in which native or recombinant F. hepatica FABPs are unable to elicit inflammatory responses. Despite the large variability among the cytokine/chemokine concentrations for each rhesus in the experimental groups, a notable lowering in the concentrations of IL-12, IL-6, IFNγ, TNFα, IP-10 or MCP-1 was observed in plasma of animals that received the Fh15-infusion compared to those that only received the E. coli-infusion. These findings support the therapeutic potential of Fh15 as an anti-inflammatory agent.

Three novel effects of a small recombinant molecule belonging to the F. hepatica fatty acid binding protein (Fh15) administered intravenously in rhesus macaques prior to an infusion with lethal amounts of live E. coli stand out from the disclosures in this application: rFh15: (1) significantly reduces bacteremia, suppresses the levels of LPS in plasma and blocks induction of CRP and PCT, which are key signature of inflammation and bacterial infection, respectively, (2) reduces production of pro-inflammatory cytokines and chemokines, and (3) prevents the immune cell facilitiates “disappearance” from blood stream. Instead, rFh15, promoted the increase of innate immune cell populations in blood, a phenomenon that seems to be similar to those observed for LPMs in the peritoneum of septic mice. This suggests a role for Fh15 in promoting a prolonged steady state or homeostasis in rhesus even in the presence of inflammatory stimuli.

Showing that a single prophylactic dose of Fh15 can suppress the bacteremia, the levels of endotoxemia as well as the levels of acute-phase proteins and a large panel of pro-inflammatory cytokines/chemokines during the early phase of sepsis in a rhesus pre-clinical sepsis model, is highly promising and will translate to a better prognosis in humans.

### Methods

### Animals

Nine healthy adult male (6-7 years-old) rhesus macaques (Macaca mulatta) aka herein “monkeys,” weighing 6.4-9.66 kg (8.1±1.08) were kindly donated by the Caribbean Primate Research Center at the University of Puerto Rico-Medical Sciences Campus. Prior to inclusion in the experiment, the monkeys received a physical examination and were tested for hematological, serological, and microbiological abnormalities. Only the animals confirmed healthy by a veterinarian were included into the experiment.

### High-Throughput Protein Expression and Purification of Fh15

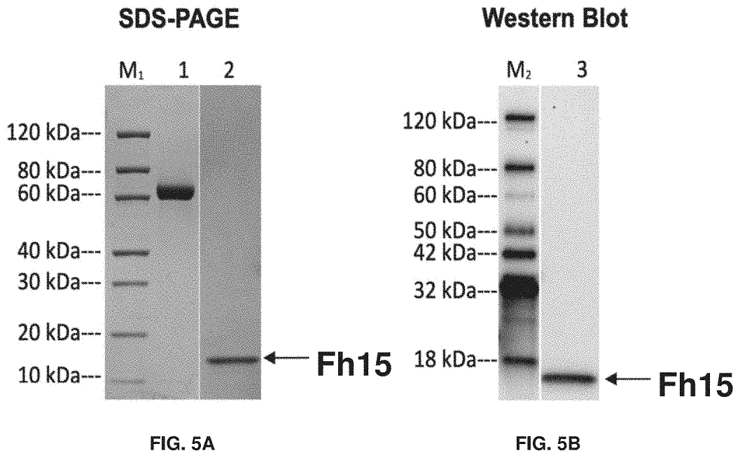

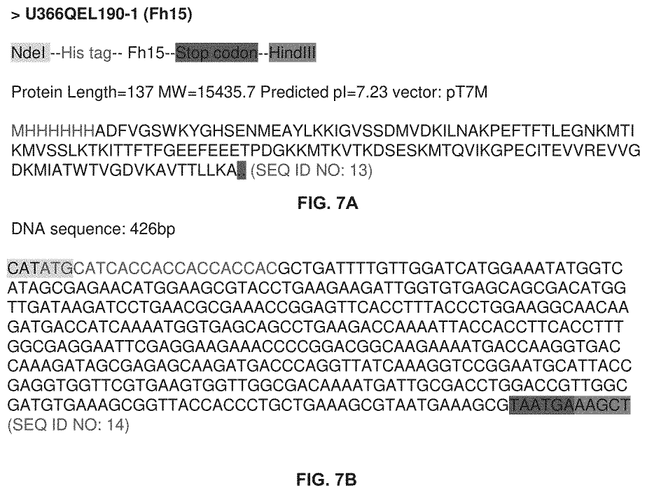

cDNA encoding full length fatty acid binding protein from F. hepatica (Fh15) (GenBank: M95291.1) was synthesized and cloned into the pT7M vector and expressed as fusion protein with His-Tag (6×His) at amino terminal in a novel bacterial expression system, using Bacillus subtilis (Genscript USA). This model offers more advantages than a previously optimized Fh15-expression system in E. coli. B. subtilis is a non-pathogenic gram-positive bacterium that does not produce LPS. Furthermore, it is not codon biased, grows faster, and has higher secretory capacity. Bacillus strain 7024E was transformed with recombinant plasmid. A single colony was inoculated into TB medium; culture was incubated at 37° C. and when the OD600 reached about 1.2, protein overexpression was induced with IPTG at 37° C. for 4 h. Cells were harvested by centrifugation and cell pellets were resuspended with lysis buffer followed by sonication. The precipitate after centrifugation was dissolved using denaturing agent. Fh15 was purified from inclusion bodies by one-step purification using Ni-column. Fh15 was stabilized in PBS containing 10% Glycerol, 0.5M NaCl, and pH 7.4 and sterilized via a 0.22 μm filter. Western blot using a mouse anti-Histidine tag monoclonal Antibody (Genscript Cat. No. A00186) was used to confirm purity of the purified protein (FIGS. 5A-5B). Purified Fh15 had endotoxin levels lower than 0.2 EU/mg measured by LAL Endotoxin Assay kit (Bioendo, Cat. No. KC64T). Protein concentration of Fh15 (3.86 mg/ml) was determined by Bradford method with BSA as a standard (ThermoFisher Cat. No. 23236). The result is shown in FIGS. 7A-7B.

### Anti-Fh15 Polyclonal Antibody and Conjugation

Polyclonal antibody against Fh15 was produced in New Zealand White rabbits by subcutaneous injections of 200 μg of protein mixed with an equal amount of complete Freund's adjuvant in the first injection, and incomplete Freund's adjuvant in the boost injections. Anti-serum had antibody titers of ˜1:100,000 when was titrated by indirect ELISA against the Fh15. Anti-Fh15 polyclonal IgG was purified by affinity chromatography using 5/5/HiTrap Protein-A HP (GE Healthcare, Piscataway, N.J.). The resulting IgG was conjugated with horseradish peroxidase (HRP, Abcam, UK, ab102890) and used to develop a Sandwich ELISA to detect the presence of circulating Fh15 in the blood of rhesus macaques.

### Escherichia coli Culture

The Escherichia coli 086a: K61 serotype used to induce sepsis was purchased from American Type Culture Collection (ATCC, 33985). Two days prior to each experiment a fresh glycerol stock was plated on a Luria Broth agar and cultured for 20 hours at 37° C. The next day a single colony was cultured in 300 ml of Luria Broth for 16-18 hours at 37° C., until it reached a concentration of 10¹⁰ CFU/ml. Afterwards, to remove free LPS the culture was harvested and washed twice with endotoxin-free PBS (0.01M phosphate buffered saline pH 7.4). The pellet was resuspended in 50 ml of endotoxin-free PBS and administrated to animals.

### Animal Preparation, and Intravenous Infusion Administration

Monkeys were fasted overnight prior to the experiment and sedated the next day with ketamine hydrochloride (100 mg/ml) intramuscularly (10 mg/kg) (Akorn-Lake Forrest, Ill.). Afterwards, they were transported to the surgical suite and anesthetized with isoflurane gas (Akorn Inc—Lake Forest, Ill.) via facemask. Then monkeys were intubated with 3.0 -3.5 mm endotracheal tubes for maintenance anesthesia and were kept intubated for the entire 8-hour period allowing them breath on their own. A 20-gauge intravenous catheter (Nipro-Osaka, Japan) was inserted in the lateral saphenous to inject the infusions prepared in isotonic saline containing 2.5% dextrose at a rate of 3.3 ml/kg/h to compensate fluid loss. To avoid discomfort, the animals remained under anesthesia during the entire experimental window of 8-hours. Monkeys were connected to a BIONET monitor that measured physiological parameters such as body temperature (BT), heart rate (HR), respiratory rate (RR), and mean arterial blood pressure (MAP) every 10 minutes. The monkeys remained under constant monitoring by veterinary staff during the complete time-course of the experiment.

The bacterial infusion consisted of 50-ml isotonic saline solution containing 10¹⁰ CFU/kg body weight (wt.) of live E. coli, which was applied at a constant rate infusion (CRI) of 0.42 ml/min over 2 h. This dose was determined to be a lethal dose for these subjects as reported elsewhere. There are no previous studies with rhesus monkeys that have been injected i.v. with proteins. Thus, a dose of 12 mg/monkey was used based on the amount of Fh15 available for the study. The 12 mg Fh15 were dissolved in 5 ml isotonic saline and applied at a constant rate infusion of 0.25 ml/min over 20 minutes. An Fh15 doses was equivalent to 1.24 to 1.87 mg/kg of body weight (average 1.48 mg/kg body wt.).

### Experimental Groups

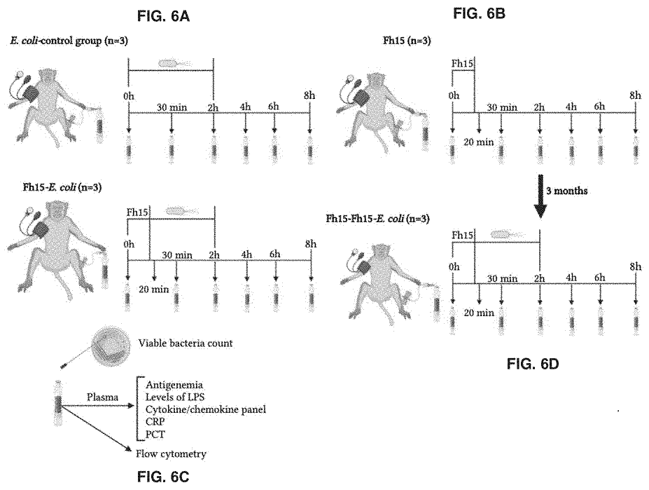

Rhesus monkeys were randomly allotted into four experimental groups designated as E. coli, Fh15, Fh15-E. coli and Fh15-Fh15-E. coli. The group designated, as E. coli was a positive control group for sepsis that only received the bacterial isotonic infusion. Following E. coli-infusion, animals were monitored for 8 h and then euthanized. The group designated as Fh15 only received an isotonic infusion of 5 ml containing 12 mg Fh15 for 20 min and after monitored for 8 h they were allowed to recover from anesthesia and returned to their original cages for 3 months. The group named Fh15-E. coli received the isotonic infusion of Fh15 for 20 min followed immediately by an E. coli infusion and were euthanized at 8 h of experimentation. The group named as Fh15-Fh15-E. coli comprised monkeys in the Fh15 group that three months later received a second infusion of Fh15 and were infected with E. coli. After infecting with E. coli monkeys were euthanized at 8 h as described above. Due to limitations of space and resources to monitor more than a monkey at a time, the experimental groups were not temporally associated. Every single monkey from each experimental group was worked separately under identical experimental conditions (FIGS. 6A-6D).

Blood samples of 5 ml were taken from the femoral vein of all animals using a 20-gauge needle into heparinized tubes (BD Vacutainer™ plastic blood collection tubes, Fisher Scientific) at 0, 30 min, 2 h, 4 h, 6 h and 8 h of experimentation. Blood samples were centrifuged at 10,000 rpm×10 minutes and the plasma was collected and stored at −20° C. in aliquots of 500 μl until further use. Prior to centrifugation, aliquots of 500 μl and 150 μl from each blood sample were allotted to determine bacteremia levels and quantifying immune cell populations by flow cytometry, respectively. After the completion of the experimental window, monkeys were euthanized with pentobarbitol solution 390 mg/ml (Med-Pharmex—Pomona, Calif.) according the AVMA Guidelines for the Euthanasia of Animals (2013). Post-mortem examination of all monkeys was conducted immediately after they were euthanized. Gross necropsy was performed and examined for any abnormalities.

### Bacteremia and Endotoxin Levels Assessment

To assess the number of viable bacteria, 500 μl of whole blood was diluted 1:1 with sterile 1×PBS, spread in a Luria Broth (LB) agar plate and culture for 20 h at 37° C. Colonies we counted and adjusted by the dilution factor. The presence of circulating levels of LPS was determined using PierceTM Endotoxin Quant kit following manufacturer's instruction (Thermo Research Scientific, US, A39553).

### C-Reactive Protein (CRP) and Procalcitonin (PCT) Concentration Determination

Plasma samples from each time point were tested for the quantification of CRP and PCT, which are characteristic inflammatory biomarkers and hallmark of bacterial infection, respectively. Quantification of CRP levels was performed at a private clinical laboratory (Martin Inc, Bayamón, PR) using an Architect c8000 Clinical Chemical Analyzer (Abbott, Ill., US). PCT assay was performed using a Human Procalcitonin ELISA kit following manufacturer's instructions (Abcam, UK, ab100630).

### Flow Cytometry

To determine the effect of Fh15 on cells of innate immune system flow cytometry was used. A total of 150 μl of anticoagulated peripheral blood was stained with surface antibodies cocktail for 30 minutes at 4° C. in the dark. Two antibody cocktails were used for the analysis, which are listed in detail in the (Table 2). After cell staining, BD lysis buffer (BD Biosciences, USA) was used to lyse the red blood cells and fix the samples for 10 minutes in the dark. Cells were washed with FACS buffer three times. Samples were stored at 4° C. in dark until the next morning for data acquisition using a MacQuant10 (Miltenyi Biotec, USA). Data was analyzed using FlowJo 10 (BD Biosciences, USA). Our gating strategy for monocytes based on their expression of CD14 and CD16 as follows: classical monocytes (CD14⁺⁺, CD16⁻), non-classical monocytes (CD14⁺, CD16⁺⁺) and intermediate monocytes (CD14⁺, CD16⁺). Neutrophils were gated based on their expression of HLA-DR, CD3⁻ and CD66 a/c/e⁺, dendritic cells (DCs) and plasmacytoid DCs based on their expression HLA-DR, CD11c⁺, CD3⁻, CD123⁺, and NK-cells based on their expression of CD3⁻, HLA-DR, CD8⁺ and NKG2a⁺.

### Sandwich ELISA for Detecting Circulating Fh15 in Plasma

A sandwich ELISA was optimized to detect circulating Fh15 in plasma from animals that received an i.v. isotonic infusion containing Fh15. The protocol followed was essentially similar to those previously developed to detect circulating antigens in human samples but using the purified rabbit anti-Fh15 IgG and the anti-Fh15 IgG-HRP conjugate described above at optimal concentrations that were determined by checkerboard titration. The anti-Fh15 IgG antibody was used as capturing antibody. It was assayed in duplicated using dilutions ranging among 0.25 to 20 m/ml in coating buffer (0.05M carbonate-bicarbonate pH 9.6). Disposable polystyrene 96-well plates (Costar, Corning, NY) were coated with 100 μl/well of each capturing antibody dilution. After an overnight incubation at 4° C. in a humid chamber the plate was washed three times with PBST and blocked with 5% skimmed milk-PBST (300 μl/well) for 1 hour at 37° C. in a humid chamber. After removing the blocking solution, undiluted plasma samples (100 μl/well) were added to the plate and incubated by 1 h 37° C. after which plates was washed three times with PBST. Anti-Fh15 IgG-HRP used as detecting antibody and was prepared at different dilutions ranging among 1:1,000 and 1:3,000 in PBST, which were added to the plate (100 μl/well) and the incubation was prolonged for 30 minutes at 37° C. in humid chamber. After another washing step, the substrate solution (50 ml 0.05M citrate-phosphate pH 5.0 30 20 mg o-phenylenediamine+20 μl H₂O₂) was added to each well (100 μl/well) and the plate was incubated at room temperature for 15 minutes in the dark. The reaction was stopped by adding 50 μl/well of 10% HCl and the optical density (OD) measured at 490 nm using a SpectraMax M3 (Molecular Devices, LLC, USA). A standard curve was generated by adding Fh15 to a negative baseline plasma sample with different concentrations of Fh15 ranging from (1 μg/ml to 61.03 pg/ml). The OD at 490 nm of spiked negative plasma sample was plotted against its known antigen protein concentration. The standard curve had an r²=0.99 using a 4 Parameter Logistic (PL) analysis. By using the standard curve, the ODs of plasma from animals used in the sepsis model were transformed into antigen concentrations (μg/ml).

### Plasma Cytokines and Chemokines Level Determination

Levels of plasma pro-inflammatory cytokines IL-6, IL-12p70, TNF-α, IFN-γ, and chemokines MCP-1, and IP-10 were determined using a Non-Human Primate Customized Multiplex (R&D Systems, Minneapolis, USA, Catalog No. FCSTM21) following manufacturer's instruction. Briefly, the assay uses magnetic microparticles pre-coated with analyte-specific antibodies, which are embedded with fluorophores at set ratios for each unique microparticle region. Microparticles, standards and plasma samples are pipetted into wells and the immobilized antibodies bind the analytes of interest. Plasma samples require 2-fold dilution with calibrator diluent provided in the kit. After washing away any unbound substances, a biotinylated antibody cocktail specific to the analytes of interest is added to each well. Following a wash to remove any unbound biotinylated antibody, streptavidin-phycoerythrin conjugate (Streptavidin-PE), which binds to the biotinylated antibody, is added to each well. After a final washing step to remove unbound Streptavidin-PE, the microparticles are resuspended in buffer and read using a Magpix (Luminex, USA) and analyzed with the Bio-Plex Data Pro Software (BioRad, Hercules, Calif.).

### Statistical Analysis

Data were analyzed by g two-way ANOVA and unpaired Student t-test using GraphPad Prism software (version 8). Data differences were considered significant at P<0.05.

### Whole Bacteria Extract Preparation

Whole bacteria extracts (WBE) were prepared from heat attenuated, normal and multidrug resistant bacterial strains (Enterococcus faecalis and Klebsiella pneumoniae). Bacteria were cultured in Luria Bertani broth (LB) for approximately 8 hours and then were killed by boiling for 15 min in a water bath.

### F. hepatica ESPs, Fh12 Purification and Antiserum Production

F. hepatica excretory-secretory products (ESPs) were obtained by in vitro maintenance technique of adult flukes freshly collected from infected livers at a local slaughterhouse as previously described. Endotoxin-free native F. hepatica fatty acid binding protein (Fh12) was purified from an adult worm extract using a previously optimized protocol that involves sequentially an ultracentrifugation step at 30,000×g, gel filtration chromatography and two preparative isoelectric focusing (IEF) steps as previously described. Polyclonal antibodies against ESPs and Fh12 were produced in rabbits by subcutaneous injections of 200 μg of protein mixed with an equal amount of complete Freund's adjuvant in the first injection, and incomplete Freund's adjuvant in the boost injections as previously described. Anti-sera had antibody titers of ˜1:102,400 or 1:51,200 when they were titrated by ELISA against the Fh12 or ESPs, respectively.

### High-Throughput Protein Expression and Purification of Recombinant F. hepatica Fatty Acid Binding Protein (Fh15)

cDNA encoding full length fatty acid binding protein from F. hepatica (Fh15) (GenBank: M95291.1) was cloned into pGEX-4T-2 vector as previously reported6. Clones expressing Fh15 as fusion protein with Schistosoma japonicum glutathione-S-transferase sequence (GST) were propagated and expressed in Escherichia coli B121 bacteria. To optimize the conditions that favor maximal expression of GST-Fh15 fusion protein, small-scale protein expression in 4-ml LB medium with 100 μg/ml ampicillin were performed. Protein expression was induced at OD600=0.7 by addition of isopropyl β-tiogalactopyranoside (IPTG) at concentrations ranging among 0.05 to 1 mM at 27° C. during 3 h. The cultures were centrifuged at 4,000 rpm for 10 min at 4° C. and then resuspended in 1-ml cold lysis buffer (0.1 M phosphate buffered saline pH 7.4 (PBS) containing 1% Triton-X-100) and then subjected to two successive cycles of freeze/thaw. Lysates were then centrifuged at 10, 000 g for 30 min, 4° C. The supernatants were collected and analyzed by 15% SDS-PAGE stained with coomassie blue and Western blot in the presence of a specific anti-GST antibody labeled with peroxidase (anti-GST-HP) as described herein.

### Scaled-Up Expression and Purification of Fh15 5

Culture of E. coli transformants containing cDNA expressing GST-Fh15 fusion protein was scaled-up to 300-ml at 37° C. Protein expression was induced at OD₆₀₀=0.7 with 0.2 mM IPTG at 27° C. for 3 h. The recombinant GST-Fh15 protein was purified in a single step by using affinity chromatography with a HP5/5 GSTrap column in an AKTA FPLC system with Unicorn software. Absorbance of eluates was monitored at 280 nm. Total cell lysates were prepared from 300-ml culture in 20 ml of lysis buffer and loaded onto column at low flow rate (˜1 ml/min). Unbound proteins were washed out with PBS. GST-tagged protein was eluted from column by washing with 10 mM Tris-HCL pH 8.0 buffer containing 10 mM reduced glutathione (GSH). GST-tagged protein was desalted against PBS using a PD-10 column (sephadex G-25). Recombinant Fh15 was excised from the GST-tag by incubation with Thrombin at a rate of 50 units thrombin by mg of fusion protein during 3 h at 4° C. with gentle agitation. After incubation, the digestion mix was reloaded onto the GSTrap column re-equilibrated with PBS and Fh15 was eluted by washing with PBS, whereas GST was eluted with 10 mM GSH as described above. The CNDB enzymatic assay was used for monitoring the efficiency of the GST-tagged protein binding to the column and recovery during the whole purification process.

### Endotoxin Removal

Endotoxins were removed from Fh15 by using polymyxin B (PMB) columns according to the manufacturer's instructions. The presence of endotoxins was assessed before and after removing endotoxins using the Chromogenic Limulus Amebocyte Lysate QCL-1000 Assay (Lonza, Walkersville, Md.) following the manufacturer's instructions. Endotoxin levels were quantified using a standard curve and reported as endotoxin units per milliliter. Protein concentration was adjusted to 1 mg/ml as determined by BCA method using a Pierce protein assay kit (Pierce, Cambridge, N.J.). Purified endotoxin-free Fh15 was stored in aliquots at −20° C. until use.

### Secondary Structure Prediction, Thermal Stability and Circular Dichroism Measurements

Secondary structure predictions of Fh15 were made using the SOPMA and Phyre2 servers available at ExPASy (Bioinformatics Resource Portal). Fifteen micrograms of Fh15 or Fh12 in 50 mM phosphate buffer, pH 8.0 were heated by 10 min at 95° C. in a water-bath and immediately tested by ELISA against the anti-Fh12 serum. Absorption spectra were recorded spectrophotometrically at 20° C. with a scan speed of 20 nm/min (200-320 nm) and compared with those dichroism (CD) measurements in the far-UV region (190-350 nm) were performed with a Jasco J-1500 CD spectrometer and protein concentrations of 0.1 mg mL-1 in 50 mM phosphate buffer, pH 8.0 in the 190-250 nm ranges at 20° C. and 95° C. using 0.1-cm paTh length cell.

### Docking Studies

The protein sequence of F. hepatica FABP1 (Fh15, Q7M4G0.3) was obtained from the UniProt database. No structures of these parasite proteins are known; thus, models were prepared using the Protein Homology/Analogy Recognition Engine (PHYRE) server, which predicts protein structure based on homology modeling. Human CD14 co-receptor structure (4GLP) was obtained from the PBD database. Proteins were docked using the ClusPro server. The top 10 balanced models were manually examined using PyMol (The PyMOL Molecular Graphics System, Version 1.5.0.4; Schrodinger), and the distances between the LPS binding site and Fh15 residues were examined and evaluated for relevance.

### Western Blot Analysis

Ten-micrograms of protein were analyzed on 15% SDS-PAGE and stained with coomassie-blue or electrotransferred to 0.45-μm nitrocellulose membranes (Bio Rad). The membranes were first blocked with 5% nonfat dry milk in PBS containing Tween-20 (PBST), then incubated 2 h at room temperature (RT) with anti-GST antibody labeled with horseradish peroxidase (HR) diluted 1:5,000 (GE Healthcare Life Sciences, USA) or incubated overnight with the anti-Fh12 or anti-ESP serum diluted 1:400 in PBST. After 3 washes with PBST, the membrane that had been incubated with the anti-GST-HP antibody was incubated at RT with the substrate solution (50-mg diaminobenzidine+100 μl H₂O₂ 30% [w/v]+100 ml PBS) until bands were visible, whereas the other membranes were incubated with the secondary antibody (goat anti-rabbit IgG-HP conjugated) diluted 1:5,000 in PBST. After another wash step, membranes were further incubated at RT with the substrate solution until bands were visible. To stop the reaction, membranes were soaked in distilled water.

### Indirect ELISA and Inhibition ELISA

Indirect ELISA was performed in disposable 96-well polystyrene plate (Costar, Corning New York), which was coated overnight with 15 μg/ml Fh15 (before or shortly after heat treatment) in 0.05 M carbonate buffer pH 9.6 as determined by checkerboard titration. After coating, the plate was washed 3 times with PBST and unbound sites in the wells were blocked with 3% non-fat dry milk diluted in PBST. After incubation for 1 h at 37° C., the plate was emptied by suction and anti-Fh12 or anti-ESP serum diluted 1:200 in PBST was added to wells (100 μl/well) in duplicate and incubated for 1 h at 37° C. after which plate was washed 3 times. Conjugate (100 μl/well peroxidase-labeled goat anti-rabbit IgG) diluted 1:5,000 in PBST-milk was added and incubated 1 h at 37° C. and then washed again 3 times. Substrate (100 μl/well of 20 μl H₂O₂, 30% [wt/v]+50 ml 0.1 M citrate buffer pH 5.0+20-mg o-phenylenediamine hydrochloride) was added and incubated in the dark at room temperature for 30 min, and the reaction was stopped with 50 μl per well of 10% HCl. Absorbance was read at 490 nm using a microplate ELISA reader (Bio Rad). In the inhibition ELISA, the anti-Fh12 serum was mixed with amounts of Fh15 or Fh12 ranging between 2.5 to 40 m/ml and incubated 1 h at 37° C. to favor antigen-antibody complex formation. Further, the antigen-serum mix was added to plate and incubated 1 h at 37° C. and the protocol continued as described above.

### Screening Assay Using THP1-Blue-CD14 Transfected Cells

To investigate the effect of Fh15 on the activation of multiple TLR-pathways, THP1-Blue-CD14 cells were used, a cell line derived from human monocytes that stably express a macrophage-specific differentiation antigen (CD14) that interact with several TLRs. THP1-Blue-CD14 cells express and respond to ligands for TLR2, TLR4, TLRS and TLR8 and are transfected with a reporter gene, secreted embryonic alkaline phosphatase (SEAP), driven by the NF-KB promoter (Invivogen, San Diego, Calif.). Upon TLR stimulation, cells activate the transcription factor and subsequently secrete SEAP, which is detected when the QUANTI-Blue™ (QB) medium is added to the culture, which turns purple in the presence of SEAP. Cells were seeded in 96-well flat-bottom plates at 1×10⁵ cells/well in 100 ∥i of RPMI supplemented with 10% heat-inactivated fetal bovine serum fetal bovine serum (FBS), 50 U/ml penicillin, 50 μg/ml streptomycin, and 2 mM L-glutamine. In the stimulation experiments cells were treated with Fh15 (2.5 to 15 μg/ml) or WBE (1×10⁸ cells/ml) and incubated at 37° C., 5% CO₂ for 24 h. Cells treated with TLR-agonist at the concentration recommended by manufacturer (Invivogen, USA) were used as activation control. The TLR-agonists used in the study were lipopolysaccharide (LPS) (1 μg/ml), heat killed Listeria monocytogenes (HKLM) (10⁸ cells/ml), flagellin (FLA) (1 μg/ml) and orthiazoloquinoline (CL075) (10 μg/ml). In the inhibition experiments, cells were cultured with Fh12 with or without heat treatment 30 min prior HKLM, LPS, FLA, CL075 or WBE stimulation. In other experiments, cells were first stimulated with LPS (1 μg/ml) for 1, 3, 6 or 12 h, after which Fh15 (10 μg) was added to the culture and incubated by additional 12 h. Afterwards, 20 μl of supernatant of each well was transferred to a clean 96-well flat-bottom plate, the QB reagent was added (180 μl/well) and the incubation prolonged for 5 h. Readings were done at 655 nm (A₆₅₅). Cells treated with PBS were used as negative control and cells incubated with polymyxin-B (PMB) (100 μM) or Chloroquin (100 μM) were used as antagonist controls. To quantify the inhibition percentage in the levels of SEAP secreted to culture media produced by Fh15, we used the formula R (%)=1−[(A−C)/(B−C)]×100, where A is the mean A₆₅₅ of three replicates obtained when cells were cultured with Fh15, and B is the mean A₆₅₅ value obtained when cells were exposed to TLR-ligands, and C is the mean A₆₅₅ of three replicates obtained when cells were stimulated with PBS.

### TLR4 Stimulation in the Presence of Anti-Fh12 Antibody

To ascertain whether the anti-Fh12 antibody could neutralize the effect of Fh15 or Fh12, THP1-Blue CD14 cells were cultured with the anti-Fh12 serum at dilutions among 1:5 to 1:400 in endotoxin-free water and 5 min later were incubated with Fh15 or Fh12 (10 μg/ml) followed by the stimulation with LPS (1 μg/ml). Cells were then incubated at 37° C., 5% CO2 for 18 h and then incubated with the QB-reagent for 5 h and absorbance were read at 655 nm as described above. As positive control, cells cultured with the antibody and stimulated with LPS were used and as negative control cells cultured only with the antibody, with PBS, Fh15 or Fh12.

### Isolation and Treatment of Mouse Bone Marrow-Derived Macrophages

BMDM were collected from femoral and tibial shafts of mice by flushing with 3 ml cold sterile PBS. The cell suspensions were passed through a sieve to remove large clumps, washed three times with sterile complete DMEM (supplemented with 20 mM L-glutamine, 1 ml penicillin and streptomycin/100 ml medium, and 10% heat-inactivated FCS; Sigma-Aldrich). Cells were adjusted to 1×10⁶ cells/well with differentiation medium (complete RPMI 1640 supplemented with 20 ng/ml M-CSF; R&D Systems) and cultured in 24-well plates (Nunc) at 37° C., 5% CO₂. On day 3 of culture, non-adherent cells were removed and the adherent cells were placed in fresh differentiation medium, and the incubation was prolonged for 7 days to cause full maturation of macrophages, which was assessed by FACS analysis and F4/80 surface Ag expression. BMDM were seeded into 24-well plates (Nunc) at 106/ml in complete DMEM and then treated with 10 μg Fh15 for 30 min before being exposed to LPS (100 ng/ml). Control cells were treated with PBS, Fh15, or LPS alone.

### Cell Viability

To determine whether the optimized Fh15 concentration affects cell viability, THP1-Blue CD14 cells and BMDM were seeded at 1×10⁶ cells/well in 96-well flat bottom plates and treated with LPS (1 μg/ml), Fh15 (10 μg/ml)+LPS (1 μg/ml) for 12 h or 24 h at 37° C. Following incubation, cell viability was assessed by adding 50 μl XTT (sodium 3′-[1-(phenylaminocarbonyl)-3,4 tetrazolium]-bis (4-methoxy-6-nitro) benzene sulfonic acid hydrate) labeling reagent (Roche Life Science, USA) to each well. The absorbance of each well was read at 480 nm.

### Quantitative Real-Time RT-PCR (qPCR)

Total RNA was extracted using an RNA isolation kit (Qiagen) kit, followed by treatment with Turbo DNA free endonuclease (Ambion, Grand Island, N.Y.) to remove contaminating genomic DNA. RNA was quantified using a Nanodrop-1000 spectrophotometer (Thermo-Scientific, USA) and a high-capacity RNA-to-cDNA kit (Applied Biosystems, Carlsbad, Calif.) to perform the reverse transcription. cDNA was amplified using a StepOne Plus Real-Time PCR system (Applied Biosystems). cDNA was equivalent to 5 ng total RNA and SYBR green PCR master Mix (Applied Biosystems). The cycling conditions were as follows: 95° C. for 15 min followed by 40 cycles of 95° C. for 15 s, 55° C. for 30 s, and 72° C. for 30 s. The primers used for each gene are listed in Table 1 below. Primer concentration was optimized and dissociation curves were generated for each primer set to verify the amplification of a single PCR product. qPCR experiments were conducted in triplicate using a StepOne Plus real-time PCR system (Applied Biosystems). The 2^{˜ΔΔCt} method was used to quantify relative gene expression using GAPDH as an internal control and expressed as fold change relative to expression in the cells stimulated with PBS. The values reported were the mean of three replicates.

### Synthesis of NIR-783-piperazima-vinyl Sulfone, Coupling to Fh15 and Tracing of Fh15-NIR-VS in Mice

NIR-783-piperazima-vinyl sulfone (NIR-VS) is a synthetic organic compound of 917,183 Da that possesses the property to excite and emit light at 490 nm and 504 nm, respectively. This product is a derivate from one previously used in immunization studies and was kindly donated by Institute of Biotechnology of University of Granada, Spain. NIR-VS was dissolved in PBS at a final concentration of 2 mg/ml and mixed with the purified Fh15 (2 mg/ml) in PBS and kept overnight at 4° C. in an orbital shaker. Free reactive groups were blocked with a molar excess of glycine in carbonate buffer at room temperature for four hours. Two mice were injected (i.p.) with 50 μg of Fh15-NIR-VS or NIR-VS alone. Animals were anesthetized (ketamine/xylazine) and the presence of Fh15 was traced in vivo at 30 min, 2 h, 12 h and 24 h after injection using an IVIS Lumina-II (Caliper LifeScience).

### Septic Shock Induction, and Serum Cytokine/Chemokine Level Measurement

Groups of 5 animals each were injected (i.p) with 7 mg/kg of body weight LPS (E. coli O111:B4, Sigma-Aldrich) and 1 h thereafter animals received a single i.p. injection of 50 μg Fh15 in PBS. Control mice were injected with PBS, Fh15 or LPS only (i.p.). Mice were sacrificed by cervical dislocation 12 h after the last injection and necropsied for collecting peritoneal exudate cells (PECs) and spleen. Animals were bled from orbital vein or cardiac puncture. Serum concentration of cytokines (TNFα, IL-1β, IL-6, IL-12p70, IL-3 and IFNγ,) and chemokines (MCP-1, KC, MIP-1a, and MIP-1b) were measured by using a Bioplex Mouse Cytokine assay (BioRad, Hercules, Calif.). Additionally, a gross macroscopically analysis was performed to assess differences between groups in pathological appearance of the peritoneal cavity.

### Peritoneal Exudate Cells (PEG) and Flow Cytometry Analysis

PECs were harvested by washing the PerC with 10 ml of cold PBS as described elsewhere. Spleens were excised into small fragments and then pressed through a strainer using the plunger end of a syringe and washed several times with PBS. Next, the suspension was washed several times by hypotonic lysis to remove erythrocytes and then resuspended in PBS by vigorous pipetting as previously described. Splenocytes or PECs were washed twice with PBS containing 2% FBS and adjusted to 1×10⁶ cells/ml. Cells were stained with different antibodies to identify macrophages. First, CD4+, CD8+, B220+, NK1.1+and dead cells were excluded by gating. Macrophages were identified as F4/80⁺ CD11b⁺ cells. The following antibodies were used in these experiments: anti-F4/80-FITC (BM8), CDllb-Pe/Cy7 (M1/70), CD38-PE (90), CD4-PACBLUE (GK1.5), CD8-PACBLUE (53-6.7) NK-1.1-PacBlue (PK-136) and CD45R-PacBlue (RA3-62B) (Biolegend, San Diego Calif.). Cells collected from animals of the same experimental group were pooled, washed twice with PBS containing 2% FBS and fixed with 1% paraformaldehyde. Cell populations were analyzed on a Miltenyi, MACS Quant Analyzer 10 instrument. Data were analyzed with FlowJo software (FlowJo, LLC). To distinguish auto fluorescence cells from cells expressing low levels of individual surface markers, upper thresholds were established for auto-fluorescence by staining samples with fluorescence-minus-one control stain sets in which a reagent for a channel of interest is omitted.

### Statistical Analysis

All data were analyzed for normality prior to statistical testing. When comparisons of the values for multiple groups were made, data were analyzed using one-way analysis of variance. For comparison of values for two groups, the Student's t-test was used using Graphpad Prism software (Prism-6). For all tests, a p value of <0.05 was deemed significant.

Although the invention has been described in conjunction with specific embodiments, it is evident that many alternatives and variations will be apparent to those skilled in the art in light of the foregoing description. Accordingly, the invention is intended to embrace all of the alternatives and variations that fall within the spirit and scope of the appended claims.

TABLE 1
Primers used in the qPCR experiments

| Murine | |
|---|---|
| Primers | Sequence (5’ to 3’) |
| IL-1β | Sense: GATCCACACTCTCCAGCTGCA |
| | (SEQ ID NO: 1) |
| | Anti-sense: CAACCAACAAGT GATAT T C T |
| | CCAT G (SEQ ID NO: 2) |
| TNFα | Sense: CATCTTCTCAAAATTCGAGTGACAA |
| | (SEQ ID NO: 3) |
| | Anti-sense: T GGGAGTAGACAAGGTACAACCC |
| | (SEQ ID NO: 4) |
| GPDH | Sense: AAGGTCATCCCAGAGCTGAA |
| | (SEQ ID NO: 5) |
| | Anti-sense: CTGCTTCACCACCTTCTTGA |
| | (SEQ ID NO: 6) |
| Human | |
| Primers | Sequence (5’ to 3’) |
| IL-1β | Sense: GCTCGCCAGTGAAATGATGG |
| | (SEQ ID NO: 7) |
| | Anti-sense: GTCCTGGAAGGAGCACTTCAT |
| | (SEQ ID NO: 8) |
| TNFα | Sense: TGGGATCATTGCCCTGTTGAG |
| | (SEQ ID NO: 9) |
| | Anti-sense: TCTAAGCTTGGGTTCCGACC |
| | (SEQ ID NO: 10) |
| β-Actin | Sense: ACAGAGCCTCGCCTTTGCCGAT |
| | (SEQ ID NO: 11) |
| | Anti-sense: TTGCACATGCCGGAGCCGTT |
| | (SEQ ID NO: 12) |

TABLE 2
Antibodies cocktail used in the flow cytometry analysis

| | | | | |
|---|---|---|---|---|
| Cell Marker | Labeling | Source | Catalog Number | References |

Cocktail-1 (NK-cells)

| | | | | |
|---|---|---|---|---|
| CD3 | PerCP-CY5.5 | BD | 552851 | Carter, D. L. et al. 1999 |
| | | | | (Cytometry, 37 (1): 41-50) |
| NKG2a | FITC | Miltenyi | 130-113-565 | Rueda, C. et al. 2016 (J. |
| | | | | Immunol. 196 (9): 3706- |
| | | | | 3715) |
| HLA-DR | VIOGREEN | Miltenyi | 130-111-795 | Edwards, J. A. et al 1986 (J. |
| | | | | Immunol. 137 (2): 490-497 |
| CD8 | PE | Biolegend | 344706 | Wakeley, M. E. et al. 2020 (J. |
| | | | | Surg. Res. 245: 610 |
| CD69 | APC/Cy7 | Biolegend | 310914 | Ahmed R. et al. 2019 (Cell, |
| | | | | 177 (6): 1583-1599 |
| CD20 | PacBlue | Biolegend | 302328 | Matzen S. M. H. et al 2018 |
| | | | | (Health Sci. Rep. 1: e90) |

Cocktail-2 (MO and DCs)

| | | | | |
|---|---|---|---|---|
| CD123 | APC | Biolegend | 306012 | Halim, T. Y. F. et al. 2018 |
| | | | | (Immunity, 48 (6): 1195- |
| | | | | 1207) |
| CD3 | PerCP-CY5.5 | BD | 552851 | Carter, D. L. et al. 1999 |
| | | | | (Cytometry, 37 (1): 41-50) |
| HLA-DR | VIOGREEN | Miltenyi | 130-111-795 | Edwards, J. A. et al 1986 (J. |
| | | | | Immunol. 137 (2): 490-497 |
| CD11c | PE/Cy7 | Biolegend | 337216 | Fournier, N. et al. 2018 |
| | | | | (MAbs 10: 651-663) |
| CD14 | FITC | Biolegend | 325604 | Walk, J. et al 2019 (Nat |
| | | | | Commun. 10: 874) |
| CD16 | PacBlue | Biolegend | 980106 | Stroncek, D. F. et al. 1991 |
| | | | | (Blood 77: 1572-1580) |
| CD66a/c/e | PE | Biolegend | 342304 | Magri G. et al. 2017 |
| | | | | (Immunity, 47: 680) |

TABLE 3
Main vital signs monitored in Rhesus macaques
during the entire 8 hours experimental course

| | | | | Value |
|---|---|---|---|---|
| | | | | at 8 h of |
| Experimental | | Physiological | Baseline | E. coli |
| Group | ID | Parameter | Value | infusion |
| E. coli | 6R1 | Body Temperature (° C.) | 35.9 | 38.6 |
| | | Heart Rate (bpm) | 108 | 192 |
| | | Mean Arterial Pressure | 58 | 25 |
| | | Respiratory Rate (rpm) | 22 | 29 |
| E. coli | 0R5 | Body Temperature (° C.) | 37.2 | 36.1 |
| | | Heart Rate (bpm) | 160 | 192 |
| | | Mean Arterial Pressure | 51 | 49 |
| | | Respiratory Rate (rpm) | 21 | 15 |
| | | Body Temperature (° C.) | 36.3 | 37 |
| E. coli | CB22 | Heart Rate (bpm) | 124 | 172 |
| | | Mean Arterial Pressure | 31 | 71 |
| | | Respiratory Rate (rpm) | 16 | 24 |
| Fh15 | MA078 | Body Temperature (° C.) | 35.7 | 36.4 |
| | | Heart Rate (bpm) | 111 | 130 |
| | | Mean Arterial Pressure | 50 | 55 |
| | | Respiratory Rate (rpm) | 21 | 21 |
| Fh15 | MA014 | Body Temperature (° C.) | 36.8 | 35.9 |
| | | Heart Rate (bpm) | 148 | 130 |
| | | Mean Arterial Pressure | 78 | 68 |
| | | Respiratory Rate (rpm) | 14 | 24 |
| Fh15 | MA035 | Body Temperature (° C.) | 37.2 | 37.2 |
| | | Heart Rate (bpm) | 140 | 142 |
| | | Mean Arterial Pressure | 53 | 70 |
| | | Respiratory Rate (rpm) | 25 | 30 |
| Fh15-Fh15- | MA078 | Body Temperature (° C.) | 35.4 | 36.3 |
| E. coli | | Heart Rate (bpm) | 112 | 128 |
| | | Mean Arterial Pressure | 51 | 49 |
| | | Respiratory Rate (rpm) | 13 | 18 |
| Fh15-Fh15- | MA014 | Body Temperature (° C.) | 33.4 | 34 |
| E. coli | | Heart Rate (bpm) | 120 | 164 |
| | | Mean Arterial Pressure | 33 | 20 |
| | | Respiratory Rate (rpm) | 23 | 20 |
| Fh15-Fh15- | MA035 | Body Temperature (° C.) | 35.6 | 34.6 |
| E. coli | | Heart Rate (bpm) | 123 | 108 |
| | | Mean Arterial Pressure | 41 | 21 |
| | | Respiratory Rate (rpm) | 20 | 5 |
| | 8Y4 | Body Temperature (° C.) | 35.9 | 37.2 |
| | | Heart Rate (bpm) | 102 | 160 |
| | | Mean Arterial Pressure | 44 | 23 |
| | | Respiratory Rate (rpm) | 22 | 14 |
| Fh15-E. coli | 0Y2 | Body Temperature (° C.) | 35.6 | 35.6 |
| | | Heart Rate (bpm) | 85 | 178 |
| | | Mean Arterial Pressure | 55 | 10 |
| | | Respiratory Rate (rpm) | 22 | 22 |
| Fh15-E. coli | IZ8 | Body Temperature (° C.) | 36.1 | 34.8 |
| | | Heart Rate (bpm) | 114 | 156 |
| | | Mean Arterial Pressure | 36 | 24 |
| | | Respiratory Rate (rpm) | 21 | 21 |

TABLE 4
Fold changes in cytokine/chemokine levels as treatment-related decreases from untreated controls

| | | | Fh15-E. coli | | | Fh15-Fh15-E. coli | | |
|---|---|---|---|---|---|---|---|---|
| Time | E. coli | | | | Fold | | | Fold |
| (hours) | Mean | ±SD | Mean | ±SD | change* | Mean | ±SD | change* |
| IFNγ | | | | | | | | |
| 0.5 h | 0 | 0 | 0 | 0 | 0 | 0 | 0 | 0 |
| 2 h | 137.57 | 117.7 | 12.19 | 4.73 | −11.28 | 9.2 | 0.001 | −14.95 |
| 4 h | 14454.63 | 11243.36 | 745.1 | 35.09 | −19.39 | 974.41 | 471.23 | −30.67 |
| 6 h | 71537.54 | 66320.46 | 3451.18 | 1223.09 | −20.72 | 9587.83 | 3301.98 | −7.46 |
| 8 h | 74824.58 | 69320.46 | 3121.45 | 601.49 | −23.97 | 10643.16 | 3421.05 | −7.03 |
| IL-6 | | | | | | | | |
| 0.5 | 170.58 | 68.58 | 73.15 | 32.77 | −2.33 | 73.78 | 28.53 | −2.31 |
| 2 h | 146204.65 | 14128.35 | 28876.59 | 5721.59 | −5.06 | 11597.51 | 4148.98 | −12.6 |
| 4 h | 164291.65 | 41362.35 | 100554.64 | 22592.85 | −1.63 | 124881.35 | 5002.85 | −0.11 |
| 6 h | 152573.7 | 45750.3 | 99850.77 | 14896.33 | −0.153 | 104109.4 | 23.0 | −1.46 |
| 8 h | 168073.7 | 49101.3 | 110231.42 | 15726.28 | −1.52 | 98478.78 | 11487.92 | −1.7 |
| IL-12 | | | | | | | | |
| 0.5 h | 137.39 | 0.001 | 6.65 | 0.009 | −20.66 | 7.48 | 5.82 | −18.36 |
| 2 h | 1717.03 | 1637.96 | 49.74 | 8.39 | −34.52 | 37.63 | 6.16 | −45.62 |
| 4 h | 51350.63 | 5123.37 | 119.43 | 8.81 | −429.9 | 162.76 | 17.11 | −315.49 |
| 6 h | 109921.18 | 10980.82 | 123.86 | 0.12 | −887.4 | 171.22 | 23.41 | −641.98 |
| 8 h | 44414.81 | 4428.19 | 115.01 | 8.84 | −386.18 | 198.77 | 16.78 | −223.44 |
| TNFα | | | | | | | | |
| 0.5 h | 764.11 | 125.11 | 32.73 | 8.31 | −23.34 | 116.36 | 111.46 | −6.57 |
| 2 h | 32698.51 | 6066.49 | 47363.74 | 3232.83 | −0.69 | 25336.86 | 714.95 | −1.29 |
| 4 h | 35388.3 | 9174.7 | 23519.15 | 1420.85 | −1.5 | 29323.96 | 2169.04 | −16.31 |
| 6 h | 29361.02 | 4367.02 | 20829.645 | 480.67 | −1.40 | 28375.92 | 1847.37 | −1.03 |
| 8 h | 12204.64 | 849.64 | 1068.54 | 164.71 | −11.42 | 10794.12 | 439.12 | −1.13 |
| IP-10 | | | | | | | | |
| 0.5 h | 404.5 | 40.5 | 370.42 | 130.59 | −5.53 | 470.52 | 135.3 | 0.86 |
| 2 h | 2804.86 | 536.86 | 1132.22 | 23.265 | −2.48 | 517.98 | 155.84 | −5.41 |
| 4 h | 17210.31 | 7366.69 | 9163.38 | 151.74 | −1.88 | 7597.30 | 930.60 | −2.26 |
| 6 h | 19789.32 | 1038.67 | 55420.98 | 4500 | −0.35 | 9672.53 | 437.46 | −2.04 |
| 8 h | 16565.13 | 6156.87 | 10192.49 | 489.34 | −1.62 | 11172.54 | 432.39 | −1.48 |
| MCP-1 | | | | | | | | |
| 0.5 h | 614.62 | 205.67 | 355.70 | 108.91 | −5.64 | 347.31 | 30.28 | −1.76 |
| 2 h | 30473.66 | 18436.33 | 14633.51 | 4079.68 | −7.469 | 8399.78 | 5757.01 | −3.62 |
| 4 h | 72428.12 | 23993.88 | 33775.11 | 3136.75 | −23.09 | 39862.24 | 4690.32 | −1.817 |
| 6 h | 64037.02 | 32384.97 | 36905.99 | 7769.24 | −8.24 | 39414.24 | 5140.32 | −1.63 |
| 8 h | 69453.48 | 26968.51 | 44032.97 | 8741.37 | −7.94 | 42713.20 | 4070.09 | −1.63 |

*Was calculated by dividing the mean concentration of each cytokine/chemokine induced by E. coli by the mean concentration induced by the Fh15-treatment at every time point, the result is expressed as negative result.

TABLE 5
Number of cells in peripheral blood from rhesus macaques treated with Fh15 alone with and
without infection with live E. coli. Each experimental group comprised three (n = 3) rhesus macaques

| | E. coli | | Fh15 | | Fh15-E. coli | | Fh15-Fh15-E. coli | |
|---|---|---|---|---|---|---|---|---|
| | Average | | Average | | Average | | Average | |
| Time | No. of | | No. of | | No. of | | No. of | |
| (hours) | cells | ±SD | cells | ±SD | cells | ±SD | cells | ± SD |

PBMC

| | | | | | | | | |
|---|---|---|---|---|---|---|---|---|
| 0 | 55634 | 23191 | 53256.3 | 4599.06 | 50663.0 | 1165.1 | 79513.6 | 12032.7 |
| 0.5 | 3766.5 | 79.5 | 36078.0 | 9465.7 | 8872.4 | 12434.2 | 61523.6 | 29280.5 |
| 2 | 2150.5 | 49.5 | 58620.3 | 16956.3 | 15685.9 | 21887.9 | 39951.0 | 29838.4 |
| 4 | 908 | 14 | 40514.0 | 41880.9 | 83821.0 | 29154.5 | 8329.3 | 4110.1 |
| 6 | 790 | 94 | 88505.0 | 32259.7 | 86605.6 | 33359.2 | 5285.0 | 2923.5 |
| 8 | 1500.5 | 69.5 | 63645.0 | 30409.1 | 73734.0 | 31958.6 | 64409.6 | 73869.7 |

CMO

| | | | | | | | | |
|---|---|---|---|---|---|---|---|---|
| 0 | 16744 | 3943 | 14576.3 | 3111.6 | 18261.6 | 8653.1 | 32592.6 | 5703.4 |
| 0.5 | 7 | 3 | 9753.6 | 3372.4 | 8574.0 | 1625.6 | 19023.3 | 145.22.8 |
| 2 | 68 | 59 | 21972.0 | 15505.4 | 22177.3 | 10542.8 | 7545.0 | 5504.6 |
| 4 | 35 | 28 | 44550.6 | 23209.7 | 45525.0 | 2145.1 | 594.3 | 483.9 |
| 6 | 18.5 | 0.5 | 51269.6 | 32871 | 55844.3 | 24580.8 | 348.0 | 408.1 |
| 8 | 50.5 | 42.5 | 32694.3 | 27591.7 | 42337.0 | 22534.5 | 45783.0 | 6158.4 |

NCMO

| | | | | | | | | |
|---|---|---|---|---|---|---|---|---|
| 0 | 3359.5 | 1968.5 | 5843.6 | 1250.1 | 3803.6 | 1609.3 | 4699.3 | 12425.3 |
| 0.5 | 26.50 | 5.5 | 2757.6 | 1015.2 | 1996.3 | 583.7 | 18535.3 | 12185.7 |
| 2 | 80 | 53 | 5331.3 | 2986.1 | 2801.0 | 1273.0 | 1120.6 | 464.5 |
| 4 | 409 | 43 | 6556.0 | 1151.5 | 3880.6 | 1219.6 | 384.0 | 322.5 |
| 6 | 426.5 | 141.5 | 8316.6 | 1513.7 | 6259.3 | 788.1 | 144.3 | 122.3 |
| 8 | 149 | 120 | 5521.0 | 608.1 | 4181.3 | 243.5 | 6272.3 | 3663.5 |

IMO

| | | | | | | | | |
|---|---|---|---|---|---|---|---|---|
| 0 | 3128 | 1524 | 1825.3 | 645.5 | 2090.3 | 1001.6 | 3415.3 | 1014.3 |
| 0.5 | 22.5 | 3.5 | 1795.6 | 1623.6 | 740.3 | 300.3 | 2990.6 | 3502.0 |
| 2 | 246.5 | 123.5 | 1875.6 | 1566.1 | 891.0 | 218.8 | 190.6 | 210.0 |
| 4 | 70.5 | 6.5 | 1141.3 | 514.8 | 764.6 | 44.9 | 15.6 | 0.94 |
| 6 | 50.5 | 31.5 | 2156.6 | 1258.7 | 1577.6 | 488.2 | 43.3 | 11.5 |
| 8 | 292 | 138 | 1608.3 | 979.9 | 1593.3 | 612.1 | 1898.3 | 2187.7 |

DCs

| | | | | | | | | |
|---|---|---|---|---|---|---|---|---|
| 0 | 5108 | 890 | 362555 | 39757.8 | 391353.0 | 184852.8 | 2912.3 | 2072.6 |
| 0.5 | 101.5 | 92.5 | 240101.6 | 45705.4 | 190228.3 | 45793.3 | 3849.6 | 1860.6 |
| 2 | 58 | 43 | 403347.6 | 78445.6 | 285749.6 | 48467.2 | 2640.0 | 1348.1 |
| 4 | 13 | 5 | 775661.6 | 50349.0 | 723955.0 | 31556.4 | 874.6 | 688.7 |
| 6 | 1 | 0.5 | 770921.0 | 53216.2 | 675481.6 | 118340.4 | 104.6 | 64.5 |
| 8 | 22.5 | 0.5 | 700466.3 | 96590.7 | 493838.6 | 187077.3 | 286.3 | 272.2 |

pDCs

| | | | | | | | | |
|---|---|---|---|---|---|---|---|---|
| 0 | 1620 | 704 | 1272.3 | 808.6 | 1162.6 | 972.2 | 2382.6 | 989.7 |
| 0.5 | 243 | 19 | 1108.0 | 702.6 | 986.3 | 504.8 | 2049.6 | 803.1 |
| 2 | 119.5 | 106.5 | 1132.6 | 644.2 | 988.6 | 590.3 | 1101.3 | 879.1 |
| 4 | 136 | 134 | 984.3 | 851.0 | 871.66 | 827.6 | 83.3 | 47.6 |
| 6 | 51 | 41 | 505.6 | 308.5 | 502.6 | 340.8 | 107.3 | 83.8 |
| 8 | 150.5 | 119.5 | 144.3 | 86.4 | 354.6 | 211.6 | 1359.3 | 908.5 |

NKC

| | | | | | | | | |
|---|---|---|---|---|---|---|---|---|
| 0 | 1248.5 | 20.5 | 6145.0 | 1352.8 | 5552.6 | 1097.5 | 9255.0 | 3204.2 |
| 0.5 | 97 | 69 | 3457.0 | 1299.9 | 3481.6 | 1433.2 | 17061.3 | 15086.2 |
| 2 | 23.5 | 6.5 | 4917.3 | 2386.2 | 4605.0 | 1731.5 | 1124.0 | 387.2 |
| 4 | 31 | 1 | 9271.6 | 1869.1 | 8079.3 | 1242.9 | 395.3 | 334.0 |
| 6 | 41.5 | 1.5 | 10087.3 | 4070.0 | 9274.0 | 3623.7 | 387.6 | 205.0 |
| 8 | 73 | 5 | 4788.0 | 2061.2 | 5982.6 | 3150.6 | 6852.0 | 6541.0 |

Neut.

| | | | | | | | | |
|---|---|---|---|---|---|---|---|---|
| 0 | 61119 | 4793 | 190.6 | 91.9 | 447.6 | 154.4 | 9625.0 | 690.0 |
| 0.5 | 38117 | 10952 | 308.6 | 208.0 | 483.0 | 211.9 | 10967.0 | 8068.0 |
| 2 | 28911 | 9318 | 409 | 244.7 | 468.3 | 156.6 | 22475.0 | 5780.0 |
| 4 | 17309 | 100 | 1046.3 | 1242.6 | 345.0 | 52.7 | 11778.3 | 6030.0 |
| 6 | 10158.5 | 9978.5 | 255.3 | 306.6 | 414.0 | 310.4 | 9003.0 | 4263.1 |
| 8 | 20259.5 | 5425.5 | 168.3 | 82.56 | 310.0 | 121.0 | 24464.6 | 15608.0 |

PBMC: peripheral blood mononuclear cells,
CMO: Classical Monocytes,
NCMO: Non-classical monocytes,
IMO: Intermediate monocytes,
DC: Dendritic cells,
pDC: Plasmacytoid Dendritic cells,
NKC: Natural Killer Cells,
Neut: Neutrophil

We claim:

1. An anti-inflammatory biotherapeutic composition comprising purified recombinant Fh15 that decreases bacteremia, endotoxemia, C-reactive protein and Procalcitonin in a mammal at risk for sepsis.

2. The rFh15 of claim 1, further defined as a fusion protein expressed with a 6×His-Tag at the carboxyl terminus in a bacterial expression system using Bacillus subtilis.

3. The composition of claim 1, wherein the mammal is a primate.

4. A method of suppressing a cytokine storm and other inflammatory markers during the early stages of sepsis in a mammal, wherein the sepsis results from Gram-negative bacteria, the method comprising:
(a) obtaining an effective dose of purified rFh15 for the mammal; and
(b) prophylactically administering the effective dose of purified rFh15 to the mammal in need thereof.

5. The method of claim 4, wherein the activation of TLR4 and the inflammatory response in the mammal is reduced compared to mammals not treated with rFh15.

6. The method of claim 4, wherein suppression of sepsis is further defined by preventing bacteremia, suppressing LPS levels in plasma, and reducing production of C-reactive protein and procalcitonin.

7. The method of claim 4, wherein innate immune cell populations in blood are reduced.

8. The method of claim 7, wherein there is an inflammatory stimulus.

9. A method of reducing sepsis in a mammal, the method comprising:
(a) obtaining a mammal with sepsis resulting from infection by Gram-negative bacteria;
(b) administering to the mammal an effective amount of recombinant Fasciola hepatica fatty acid binding protein (Fh15) during the early stages of infection.

10. The method of claim 9, wherein the recombinant Fh15 is administered intravenously.


SEQUENCE LISTING (14 sequences)

SEQ ID NO: 1 - DNA, 21 bp, Artificial Sequence
  Description of Artificial Sequence Synthetic PCR Murine Primer
gatccacactctccagctgca

SEQ ID NO: 2 - DNA, 25 bp, Artificial Sequence
  Description of Artificial Sequence Synthetic PCR Murine Primer
caaccaacaagtgatattctccatg

SEQ ID NO: 3 - DNA, 25 bp, Artificial Sequence
  Description of Artificial Sequence Synthetic PCR Murine Primer
catcttctcaaaattcgagtgacaa

SEQ ID NO: 4 - DNA, 23 bp, Artificial Sequence
  Description of Artificial Sequence Synthetic PCR Murine Primer
tgggagtagacaaggtacaaccc

SEQ ID NO: 5 - DNA, 20 bp, Artificial Sequence
  Description of Artificial Sequence Synthetic PCR Murine Primer
aaggtcatcccagagctgaa

SEQ ID NO: 6 - DNA, 20 bp, Artificial Sequence
  Description of Artificial Sequence Synthetic PCR Murine Primer
ctgcttcaccaccttcttga

SEQ ID NO: 7 - DNA, 20 bp, Artificial Sequence
  Description of Artificial Sequence Synthetic PCR Human Primer
gctcgccagtgaaatgatgg

SEQ ID NO: 8 - DNA, 21 bp, Artificial Sequence
  Description of Artificial Sequence Synthetic PCR Human Primer
gtcctggaaggagcacttcat

SEQ ID NO: 9 - DNA, 21 bp, Artificial Sequence
  Description of Artificial Sequence Synthetic PCR Human Primer
tgggatcattgccctgttgag

SEQ ID NO: 10 - DNA, 20 bp, Artificial Sequence
  Description of Artificial Sequence Synthetic PCR Human Primer
tctaagcttgggttccgacc

SEQ ID NO: 11 - DNA, 22 bp, Artificial Sequence
  Description of Artificial Sequence Synthetic PCR Human Primer
acagagcctcgcctttgccgat

SEQ ID NO: 12 - DNA, 20 bp, Artificial Sequence
  Description of Artificial Sequence Synthetic PCR Human Primer
ttgcacatgccggagccgtt

SEQ ID NO: 13 - protein, 137 aa, Artificial Sequence
  Description of Artificial Sequence Synthetic polypeptide
MHHHHHHADFVGSWKYGHSENMEAYLKKIGVSSDMVDKILNAKPEFTFTLEGNKMTIKMVSSLKTKITTFTFGEEFEEETPDGKKMTKVTKDSESKMTQVIKGPECITEVVREVVGDKMIATWTVGDVKAVTTLLKA

SEQ ID NO: 14 - DNA, 436 bp, Artificial Sequence
  Description of Artificial Sequence Synthetic polynucleotide
catatgcatcaccaccaccaccacgctgattttgttggatcatggaaatatggtcatagcgagaacatggaagcgtacctgaagaagattggtgtgagcagcgacatggttgataagatcctgaacgcgaaaccggagttcacctttaccctggaaggcaacaagatgaccatcaaaatggtgagcagcctgaagaccaaaattaccaccttcacctttggcgaggaattcgaggaagaaaccccggacggcaagaaaatgaccaaggtgaccaaagatagcgagagcaagatgacccaggttatcaaaggtccggaatgcattaccgaggtggttcgtgaagtggttggcgacaaaatgattgcgacctggaccgttggcgatgtgaaagcggttaccaccctgctgaaagcgtaatgaaagcgtaatgaaagct